(19) United States
(12) Patent Application Publication
(10) Pub. No.: US 2024/0044832 A1
(43) Pub. Date: Feb. 8, 2024

(54) METHODS FOR DIAGNOSING CANCER BASED ON VOLATILE ORGANIC COMPOUNDS IN BLOOD AND URINE SAMPLES

(71) Applicant: TECHNION RESEARCH AND DEVELOPMENT FOUNDATION LTD., Haifa (IL)

(72) Inventors: Hossam HAICK, Haifa (IL); Reef EINOCH AMOR, Haifa (IL); Yoav BROZA, Haifa (IL)

(21) Appl. No.: 18/268,638

(22) PCT Filed: Dec. 21, 2021
(86) PCT No.: PCT/IL2021/051515

§ 371 Date: Jun. 21, 2023

Related U.S. Application Data
(60) Provisional application No. 63/128,188, filed on Dec. 21, 2020.

(51) Int. Cl. G01N 27/327; G01N 1/22; G16H 50/20; G16H 50/70

(52) U.S. Cl. CPC G01N 27/3275; G01N 1/2226; G16H 50/20; G16H 50/70; G01N 2001/2229

(57) ABSTRACT

The present invention provides methods of diagnosing cancer in a test subject, comprising exposing an array of chemically sensitive sensors comprising a material selected from the group consisting of conductive nanostructures coated with an organic coating, a conducting polymer and a conductive polymer composite, to a blood sample and a urine sample obtained from the test subject, and analyzing output signals of the chemically sensitive sensors upon exposure of the array to the blood sample and the urine sample. The array of the chemically sensitive sensors can be a part of a portable medical device. Further provided is a method of diagnosing cancer in a test subject, comprising measuring and analyzing levels of a set of volatile organic compounds (VOCs) in a blood sample and a urine sample obtained from the test subject.

### FIELD OF THE INVENTION

The present invention relates to methods for the diagnosis, prognosis, monitoring or differentiating between various types of cancer by analyzing volatile organic compounds found in blood and urine samples and by using chemically sensitive sensors.

### BACKGROUND OF THE INVENTION

Cancer accounts for most morbidity and mortality worldwide, with about 15 million new cases and 9.6 million cancer-related deaths in 2018. Although there can be a higher incidence in developed countries, over 60% of the world's cancer cases occur in developing countries, which can account for about 70% of the cancer deaths. The predicted global cancer burden is expected to exceed 21 million new cancer cases annually by 2030, making it a significant economic burden and a matter that necessitates major research worldwide.

In 2010, the total annual economic cost of cancer through healthcare expenditure and loss of productivity was estimated at US$1.16 trillion. When identified early, cancer is more likely to respond to effective treatment and can result in a greater probability of surviving, less morbidity, and lower costs. Currently there are several diagnostic methods, such as imaging, biopsy, and biomarkers in biofluids. However, these methods still have major limitations.

Most of the available diagnostic methods are expensive, time consuming (from days up to several weeks) and require laboratory work and expensive equipment. Moreover, the sensitivity of some of the most common imaging techniques currently used to diagnose cancer depends mostly on tumor size, wherein said techniques are costly, and/not suitable for widespread screening. To date, histological biopsy remains the gold standard diagnostic in cancer, although it is invasive, risky, time-consuming and expensive. Therefore, there is an urgent need for inexpensive and non-invasive diagnostics that would allow: 1) early detection of cancer; 2) personalized therapy according to population bio-specification; and 3) efficient treatment monitoring.

Identification of cancer biomarkers can clearly improve cancer survival rates and reduce the overall morbidity.

Circulating Tumor Cells (CTCs) have been shown to predict cancer progression and survival in metastatic disease, even in early-stage cancer patients. CTCs, regarded as a “liquid biopsy”, is minimally invasive and can be considered more effective than existing standards in monitoring the progression of the disease and its treatments in real-time. Molecular analyses of CTCs can be used for real-time monitoring of disease aggressiveness and treatment response. Pathological changes may also be detected in the urinary components, which can be obtained non-invasively, in large amounts, and at minimum cost.

Volatile Organic Compounds (VOCs) are organic compounds that have a high vapor pressure at room-temperature conditions. Analysis of VOCs is an innovative approach, which is non- or minimally-invasive, fast and potentially inexpensive. VOCs analysis allows monitoring of human body chemistry related to health and morbid conditions. VOCs are generated in the microenvironment of the cells; following their production they are emitted and therefore can be found in different bodily fluids, including, but not limited to: (i) damaged/infected cells and/or their microenvironment, (ii) blood, (iii) breath, (iv) skin, (v) urine, (vi) feces, and/or (vii) saliva. Consequently, VOCs may be collected from the headspace of these matrices and examined. VOCs have been extensively studied, especially in breath analysis, where the findings suggest that they can serve as biomarkers of different systemic diseases. Although breath analysis of Volatile Organic Compounds (VOCs) has the potential to overcome the limitations of histological biopsy, it is also prone to several potential problems. For example, different VOCs with the same concentration in exhaled breath may show very different concentrations in fat and blood (up to a factor of 10), due to the fact that different materials have different blood:air and fat:air partition coefficients. VOCs with high blood:air partition coefficients may show lower volatility and may not be detected in breath. Therefore, for some diseases and VOCs analyzing blood or urine VOCs would be the best approach.

In the literature there are several reports on cancer related VOCs which can be detected directly from bodily fluids; however, these reports are specific for a single disease type and are mainly focused on Gas-Chromatography-lined Mass-Spectrometry (GC-MS) analysis, which is time consuming, expensive and requires trained personnel.

WO 2000/041623 discloses a process for determining the presence or absence of a disease, particularly breast or lung cancer, in a mammal, comprising collecting a representative sample of alveolar breath and a representative sample of ambient air, analyzing the samples of breath and air to determine content of n-alkanes having 2 to 20 carbon atoms, inclusive, calculating the alveolar gradients of the n-alkanes in the breath sample in order to determine the alkane profile, and comparing the alkane profile to baseline alkane profiles calculated for mammals known to be free of the disease to be determined, wherein finding of differences in the alkane profile from the baseline alkane profile being indicative of the presence of the disease.

WO 2012/009578 is directed to a diagnostic method for cancer detection based on miRNA levels in the patient's blood sample. The method based on oligonucleotide detection by using nanopore technology with a probe containing a complementary sequence to the target oligonucleotide and a terminal extension at the probe's 3′ terminus, 5′ terminus, or both termini.

WO 2012/155118 is directed to guided-mode resonance (GMR) sensor systems, and in particular to a GMR sensor that can be used to simultaneously detect an array of analytes and can provided in a portable configuration.

WO 2017/155570 is directed to methods of producing a circulating analyte profile of a subject. The methods include contacting a blood sample from a subject with a panel of probes for specific binding to analytes and detecting the presence or absence of binding of the analytes to probes of the panel of probes. Also provided are sensor devices including a panel of capture probes and useful, e.g., for practicing the methods of the present disclosure.

WO 2018/004414 is directed to a method for cancer detection and screening, based on analysis of Volatile Organic Compounds (VOCs) emitted by certain cancerous tumors. The device and method provide high sensitivity and specificity analyses. The sample to be analyzed may be e.g., blood or blood plasma. In one aspect, the invention is directed towards detection of or screening for gynecological cancers, e.g. ovarian cancer.

The US Application No. 2019/0204321 is directed to a high-sensitivity liquid field-effect sensor for colon cancer, applicable to a sample such as blood or stool. The sensor according to one aspect enables ultra-high precision/low-concentration detection of colon cancer biomarkers, thereby having an effect of enabling early diagnosis of colon cancer even with a very small amount of a sample.

WO 2019/053414 is directed to biomarkers, and to biological markers for diagnosing various conditions, including cancer. In particular, to the use of these compounds as diagnostic and prognostic markers in assays for detecting cancer, such as pancreatic cancer and/or colorectal cancer, and corresponding methods of detection. WO 2019/053414 is further directed to methods of determining the efficacy of treating these diseases with a therapeutic agent, and apparatus for carrying out the assays and methods.

WO 2010/079490 to one of the inventors of the present application discloses a sensor array for detecting biomarkers for cancer in breath samples. The sensor array is based on 2D films or 3D assemblies of conductive nanoparticles capped with an organic coating wherein the nanoparticles are characterized by a narrow size distribution.

There remains an unmet need for a non-invasive and time- and cost-effective technique for diagnosing cancer, which would also allow to distinguish between disease specific subtypes, assess disease activity and prognosis, as well as predict response to therapeutics in patients with cancer.

### SUMMARY OF THE INVENTION

The present invention provides methods for diagnosing, staging, monitoring or prognosing cancer in a subject, which also allow differentiation between different cancer types. The methods of the present invention may further enable the assessment or prediction of the course of the disease, as well as the prediction of the response to a treatment regimen.

The present invention provides a diagnostic method, which is based, in some embodiments, on a small mobile device that can detect cancer-associated internal molecular alterations in the headspace of small samples of urine and blood samples, by being sensitive towards volatile organic compounds released from or in response to cancer cells incidence. Alternatively, the VOCs from the urine and blood samples' headspace can be assessed by established analytical systems, such as, for example, GC-MS.

The present invention is based in part on a surprising finding that analyzing the headspace of both blood and urine samples allows to increase cancer diagnosis efficiency. In particular, it has been shown that the combined approach provided higher values of discrimination accuracy, sensitivity and specificity, as compared to the individual analysis of the urine and blood samples, when diagnosing gastric, kidney cancer and lung cancer by means of GC-MS. Diagnosis accuracy and sensitivity were also enhanced when analyzing both the blood sample and the urine sample by chemically sensitive sensors.

It has further been unexpectedly found that a single chemically sensitive sensor comprising gold nanoparticles coated with octadecanethiol was able to efficiently discriminate between different cancer stages in both blood and urine samples with high statistical significance. The inventors have further discovered specific combinations of the chemically sensitive sensors, as well as particular sets of VOCs, which enable reliable diagnosis of multiple types of cancer.

In one aspect, the present invention provides a method of diagnosing cancer in a test subject, comprising contacting a portable device with a blood sample and a urine sample obtained from the test subject, wherein the portable device comprises an array of chemically sensitive sensors comprising a material selected from the group consisting of conductive nanostructures coated with an organic coating, a conducting polymer and a conductive polymer composite, and analyzing output signals of the chemically sensitive sensors upon exposure of the array to the blood sample and the urine sample. Contacting comprises drawing an aliquot of a headspace of the blood sample and an aliquot of a headspace of the urine sample into the device and exposing the array to each aliquot individually. Analyzing comprises using a model based on a database of response patterns of the array of chemically sensitive sensors to control samples comprising blood samples and urine samples obtained from patients having the cancer and healthy subjects. The cancer, which can be diagnosed by said method is selected from the group consisting of kidney cancer, gastric cancer, lung cancer, breast cancer, pancreatic cancer, ovarian cancer, colon cancer, head and neck cancer, prostate cancer, and combinations thereof.

According to some embodiments, the conductive nanostructures coated with an organic coating are selected from gold nanoparticles (GNPs) coated with a thiol or a disulfide and single walled carbon nanotubes (SWCNTs) coated with polycyclic aromatic hydrocarbon (PAH). In some embodiments, the thiol is selected from the group consisting of dodecanethiol, 4-tert methyl-benzenethiol, 2-ethylhexanethiol, decanethiol, 4-chlorobenzenemethanethiol, 3-ethoxytiophenol, hexanethiol, octadecanethiol, and combinations thereof. In some embodiments, the polycyclic aromatic hydrocarbon comprises hexa-perihexabenzocoronene or a derivative thereof.

The conducting polymer can be selected from the group consisting of diketopyrrolopyrrole-naphthalene copolymer (PDPP-TNT), polydiketopyrrolopyrrole, polyaniline (PANI), polythiophene, poly(3,4-ethylenedioxythiophene)-poly(styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof.

In some embodiments, the conductive polymer composite comprises a polymer selected from the group consisting of a disulfide polymer, a methacrylate polymer, a polyethyleneimine polymer, combinations and derivatives thereof, wherein said polymer is mixed with a carbon powder. In certain embodiments, the conductive polymer composite is selected from the group consisting of carbon black/poly(propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

In some exemplary embodiments, the array comprises eight chemically sensitive nanosensors comprising gold nanoparticles coated with dodecanethiol, 4-tert methyl-benzenethiol, 2-ethylhexanethiol, decanethiol; 4-chlorobenzenemethanethiol, 3-ethoxytiophenol, hexanethiol, and octadecanethiol.

According to some embodiments, the array of chemically sensitive sensors is sealed within the portable device from external atmosphere.

According to some embodiments, the portable device further comprises a sample inlet, a cannula, and a pipe, wherein the pipe is connected to the cannula at one end and to the sample inlet at another end. In further embodiments, drawing an aliquot of a headspace of the blood sample and an aliquot of a headspace of the urine sample comprises inserting the cannula into a vial comprising the respective sample and pumping the headspace into the portable device. The pumping rate can range from about 30 μl/s to about 3300 μl/s. The pumping can be performed for a period ranging from about 0.5 s to about 5 s. In certain embodiments, the array is exposed to the aliquot of the headspace for a period ranging from about 5 s to about 120 s. In certain embodiments, the array is exposed to each aliquot of the headspace for a period ranging from about 5 s to about 120 s.

According to some embodiments, the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal.

According to some embodiments, the cancer is selected from the group consisting of kidney cancer, gastric cancer, and lung cancer.

According to some embodiments, the model is developed by using at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

According to some currently preferred embodiments, contacting the portable device with each one of the blood sample and the urine sample provides enhanced accuracy, sensitivity and/or specificity of the diagnosing as compared to contacting with only one of said blood sample and urine sample.

According to some embodiments, the method further comprises contacting the portable device with a body tissue sample obtained from the test subject.

In another aspect, there is provided a method of diagnosing cancer in a test subject, comprising exposing an array of chemically sensitive sensors comprising a material selected from the group consisting of conductive nanostructures coated with an organic coating, a conducting polymer and a conductive polymer composite, to a blood sample and a urine sample obtained from the test subject, wherein the array comprises gold nanoparticles coated with octadecanethiol, and analyzing output signals of the chemically sensitive sensors upon exposure of the array to the blood sample and urine sample. Analyzing comprises using a model based on a database of response patterns of the array of the chemically sensitive sensors to control samples comprising blood and urine samples obtained from patients having the cancer and healthy subjects. The cancer, which can be diagnosed by said method is selected from the group consisting of kidney cancer, gastric cancer, lung cancer, breast cancer, pancreatic cancer, ovarian cancer, colon cancer, head and neck cancer, prostate cancer, and combinations thereof.

According to some embodiments, the array further comprises gold nanoparticles coated with an organic coating selected from the group consisting of tert-dodecanethiol, butanethiol, 4-cholorobenzenemethanthiol, 4-tert butylbenzenethiol, 2-naphthalenethiol, 2-nitro-4-(trifluoromethyl)benzenethiol, dodecanethiol, decanethiol, 3-ethoxythiophenol, benzylmercaptan, hexanethiol, 2-ethylhexanethiol, 1,6-hexanedithiol, butanethiol, dibutyl disulfide, and combinations thereof. In certain embodiments, the array further comprises gold nanoparticles coated with decanethiol and gold nanoparticles coated with 3-ethoxythiophenol. According to some exemplary embodiments, the array further comprises gold nanoparticles coated with decanethiol, gold nanoparticles coated with 3-ethoxythiophenol, gold nanoparticles coated with dodecanethiol, gold nanoparticles coated with 2-ethylhexanethiol, and gold nanoparticles coated with tert-dodecanethiol.

The array can further comprise single walled carbon nanotubes (SWCNTs) coated with a polycyclic aromatic hydrocarbon (PAH) or a derivative thereof selected from the group consisting of hexa-peri-hexabenzocoronene (HBC) molecules. HBC molecules can be unsubstituted or substituted by any one of methyl ether, 2-ethyl-hexyl (HBC-C6,2), 2-hexyldecyl (HBC-C10,6), 2-decyltetradecyl (HBC-C14,10), and dodecyl (HBC-C12). In certain embodiments, the PAH is crystal hexakis(n-dodecyl)-peri-hexabenzocoronene (HBC-C12).

According to some embodiments, the array further comprises a conducting polymer selected from the group consisting of diketopyrrolopyrrole-naphthalene copolymer (PDPP-TNT), polydiketopyrrolopyrrole, polyaniline (PANI), polythiophene, poly (3,4-ethylenedioxythiophene)-poly (styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof. In certain embodiments, said conducting polymer is diketopyrrolopyrrole-naphthalene.

The array can further comprise a conductive polymer composite selected from the group consisting of a disulfide polymer, a methacrylate polymer, a polyethyleneimine polymer, combinations and derivatives thereof, wherein said polymer is mixed with a carbon powder. In certain embodiments, the array further comprises carbon black/poly (propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

According to some embodiments, the cancer is selected from the group consisting of kidney cancer, gastric cancer, and lung cancer.

According to some embodiments, the model is developed by using at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

According to some embodiments, the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal.

According to some currently preferred embodiments, exposing the array to each one of the blood sample and the urine sample provides enhanced accuracy, sensitivity and/or specificity of the diagnosing as compared to exposing to only one of said blood sample and urine sample.

According to some embodiments, the method further comprises exposing the array of the chemically sensitive sensors to a body tissue sample obtained from the test subject.

In yet another aspect, there is provided a method of diagnosing cancer in a test subject, comprising measuring and analyzing levels of a set of volatile organic compounds (VOCs) in a blood sample and a urine sample obtained from the test subject. The set of VOCs comprises at least five VOCs selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, and 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, 3-methyl butanal. Analyzing comprises using a model based on a database of levels of the set of VOCs in control samples comprising blood and urine samples obtained from patients having the cancer and healthy subjects. The cancer is selected from the group consisting of kidney cancer, gastric cancer, lung cancer, breast cancer, pancreatic cancer, ovarian cancer, colon cancer, head and neck cancer, prostate cancer, and combinations thereof.

According to some embodiments, the set of VOCs comprises at least five VOCs selected from the group consisting of hexane, 3-methyl butanal, pentanal, 2.3-dihydro furan, hexanal, 1,3,5-trimethyl cyclohexane, 2,4-dimethyl1-heptene, 2,4-dimethyl decane, 4,7-dimethyl undecane, 2,4-dimethyl heptane, 4-methyl octane, 2-ethyl 1-hexanol, dodecane, 5-ethyl,2-methyl octane, and combinations thereof.

According to some embodiments, the set of VOCs comprises at least five VOCs selected from the group consisting of 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydro furan, 2,3,5 trimethyl hexane, 2-pentyl furan, and 3-methyl butanal.

According to certain embodiments, the set of VOCs comprises 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydrofuran, 2,3,5 trimethyl hexane, 2-pentyl furan, and 3-methyl butanal.

According to some embodiments, the set of VOCs comprises at least five VOCs selected from the group consisting of hexane, 3-methyl butanal, pentanal, 2.3-dihydro furan, hexanal, 1,3,5-trimethyl cyclohexane, 2,4-dimethyl1-heptene, 2,4-dimethyl decane, 4-methyl octane, and 5-ethyl,2-methyl octane.

According to some embodiments, the set of VOCs comprises at least five VOCs selected from the group consisting of 3-methyl butanal, pentanal, hexanal, 2,3-dihydro furan, 2,4-dimethyl decane, dodecane, 2-ethyl hexanol, 5-ethyl-2-methyl octane.

According to some embodiments, the model is developed by using at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

According to some embodiments, measuring the levels of a set of VOCs comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), and Proton Transfer Reaction Mass-Spectrometry (PTR-MS). In certain embodiments, measuring the levels of a set of VOCs comprises the use of Gas-Chromatography-Mass Spectrometry (GC-MS) combined with In-tube Extraction (ITEX).

According to some embodiments, the cancer is selected from the group consisting of kidney cancer, gastric cancer, and lung cancer.

According to some currently preferred embodiments, analyzing each one of the blood sample and the urine sample provides enhanced accuracy, sensitivity and/or specificity of the diagnosing as compared to analyzing only one of said blood sample and urine sample.

According to some embodiments, the method further comprises analyzing levels of the set of VOCs in a body tissue sample obtained from the test subject.

In yet another aspect, there is provided a method of diagnosing cancer in a test subject, comprising contacting a portable device with a blood sample or a urine sample obtained from the test subject, wherein the portable device comprises an array of chemically sensitive sensors comprising a material selected from the group consisting of conductive nanostructures coated with an organic coating, a conducting polymer and a conductive polymer composite, and analyzing output signals of the chemically sensitive sensors upon exposure of the array to the blood sample or urine sample. Contacting comprises drawing an aliquot of a headspace of the blood sample or an aliquot of a headspace of the urine sample into the device and exposing the array to said aliquot. Analyzing comprises using a model based on a database of response patterns of the array of chemically sensitive sensors to control samples comprising blood samples or urine samples obtained from patients having the cancer and healthy subjects. The cancer, which can be diagnosed by said method is selected from the group consisting of kidney cancer, gastric cancer, lung cancer, breast cancer, pancreatic cancer, ovarian cancer, colon cancer, head and neck cancer, prostate cancer, and combinations thereof.

According to some embodiments, the conductive nanostructures coated with an organic coating are selected from gold nanoparticles (GNPs) coated with a thiol or a disulfide and single walled carbon nanotubes (SWCNTs) coated with polycyclic aromatic hydrocarbon (PAH). In some embodiments, the thiol is selected from the group consisting of dodecanethiol, 4-tert methyl-benzenethiol, 2-ethylhexanethiol, decanethiol, 4-chlorobenzenemethanethiol, 3-ethoxytiophenol, hexanethiol, octadecanethiol, and combinations thereof. In some embodiments, the polycyclic aromatic hydrocarbon comprises hexa-perihexabenzocoronene or a derivative thereof.

The conducting polymer can be selected from the group consisting of diketopyrrolopyrrole-naphthalene copolymer (PDPP-TNT), polydiketopyrrolopyrrole, polyaniline (PANI), polythiophene, poly(3,4-ethylenedioxythiophene)-poly(styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof.

In some embodiments, the conductive polymer composite comprises a polymer selected from the group consisting of a disulfide polymer, a methacrylate polymer, a polyethyleneimine polymer, combinations and derivatives thereof, wherein said polymer is mixed with a carbon powder. In certain embodiments, the conductive polymer composite is selected from the group consisting of carbon black/poly(propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

In some exemplary embodiments, the array comprises eight chemically sensitive nanosensors comprising gold nanoparticles coated with dodecanethiol, 4-tert methyl-benzenethiol, 2-ethylhexanethiol, decanethiol; 4-chlorobenzenemethanethiol, 3-ethoxytiophenol, hexanethiol, and octadecanethiol.

According to some embodiments, the array of chemically sensitive sensors is sealed within the portable device from external atmosphere.

According to some embodiments, the portable device further comprises a sample inlet, a cannula, and a pipe, wherein the pipe is connected to the cannula at one end and to the sample inlet at another end. In further embodiments, drawing an aliquot of a headspace of the blood sample or an aliquot of a headspace of the urine sample comprises inserting the cannula into a vial comprising the sample and pumping the headspace into the portable device. The pumping rate can range from about 30 μl/s to about 3300 μl/s. The pumping can be performed for a period ranging from about 0.5 s to about 5 s. In certain embodiments, the array is exposed to said aliquot for a period ranging from about 5 s to about 120 s.

According to some embodiments, the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal.

According to some embodiments, the cancer is selected from the group consisting of kidney cancer, gastric cancer, and lung cancer.

According to some embodiments, the model is developed by using at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

According to some embodiments, the method comprises contacting the portable device with each one of the blood sample and the urine sample.

According to some embodiments, the method further comprises contacting the portable device with a body tissue sample obtained from the test subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B: Hierarchical clustering (FIG. 1A) and corresponding constellation plot (FIG. 1B) of the combined data from blood and urine samples together based on the identified VOCs. The dendrogram represents the relative distance between samples based on the VOC data. Constellation (tree) plot arranges the sample clusters, the lines represent membership in a cluster. The length of a line between cluster joins approximates the distance between the clusters that were joined. Rows represent the different clinical samples: C=control; KC=kidney cancer; GC=gastric cancer; LC=lung cancer; FG=fibro gastroscopy.

FIGS. 2A-2F: Violin plot of the VOCs which provided the lowest p value, for the blood data based on the VOCs abundance, including the relative abundance of Methyl butanal (FIG. 2A), the relative abundance of Dodecane (FIG. 2B), the relative abundance of Hexane (FIG. 2C), the relative abundance of Dihydro Furan (FIG. 2D), the relative abundance of Unknown (6) VOC (FIG. 2E) and the relative abundance of Hexanal (FIG. 2F).

FIGS. 3A-3F: Violin plot of the VOCs which provided the lowest p value, for the urine data based on the average VOCs abundance, including the relative abundance of Dodecane (FIG. 3A), the relative abundance of Octane (FIG. 3B), the relative abundance of Ethanone (FIG. 3C), the relative abundance of Pentanone (FIG. 3D), the relative abundance of 2-Heptanone (FIG. 3E) and the relative abundance of Unknown (4) VOC (FIG. 3F).

FIG. 4: Test sets receiver operating characteristic curve (ROC) for the three models discriminating cancer from non-cancer volunteers. Each ROC is summarized by its median value (solid curve) and an envelope representing the minimal and maximal value obtained within the outer loop of the nested cross validation.

FIG. 5A-5C: Distribution of the output probabilities for three different stages of cancer, including the distribution of the output probabilities for cancer of stage 1 among 16 patients (FIG. 5A), the distribution of the output probabilities for cancer of stage 2 among 52 patients (FIG. 5B) and the distribution of the output probabilities for cancer of stage 3 among 18 patients (FIG. 5C).

FIG. 6A: Photograph of the portable device.

FIG. 6B: Photograph of a chip on which the sensor array is disposed within the portable device.

FIG. 6C: Micrograph of the sensor array.

FIG. 6D: Photograph showing a step of drawing an aliquot of the headspace of a blood sample into the portable device.

FIGS. 7A-7C: DFA plots of the first canonical variable (CV1) calculated from the response of sensors from mobile device to the headspace of blood samples, including samples originated from non-cancer (Control) and Gastric cancer patients (FIG. 7A), samples originated from non-cancer (Control) and Kidney cancer patients (FIG. 7B) and samples originated from non-cancer (Control) and Lung cancer patients (FIG. 7C).

FIGS. 8A-8C: DFA plots of the first canonical variable (CV1) calculated from the response of sensors from mobile device to the headspace of blood samples, including samples originated from Kidney and Lung cancer patients (FIG. 8A), samples originated from Gastric and Kidney cancer patients (FIG. 8B) and samples originated from Gastric and Lung cancer patients (FIG. 8C).

FIGS. 9A-9C: DFA plots of the first canonical variable (CV1) calculated from the response of sensors from mobile device to the headspace of urine samples, including samples originated from non-cancer (Control) and Gastric cancer patients (FIG. 9A), samples originated from non-cancer (Control) and Kidney cancer patients (FIG. 9B) and samples originated from non-cancer (Control) and Lung cancer patients (FIG. 9C).

FIGS. 10A-10C: DFA plots of the first canonical variable (CV1) calculated from the response of sensors from mobile device to the headspace of urine samples, including samples originated from Kidney and Lung cancer patients (FIG. 10A), samples originated from Gastric and Kidney cancer patients (FIG. 10B) and samples originated from Gastric and Lung cancer patients (FIG. 10C).

### DETAILED DESCRIPTION OF THE INVENTION

The present invention is directed to methods for diagnosing, monitoring or prognosing cancer including, inter alia, kidney cancer, gastric cancer, lung cancer, breast cancer, pancreatic cancer, ovarian cancer, colon cancer, head and neck cancer, prostate cancer, in a subject. The methods of the present invention further provide the differentiation between said types of cancer, disease states, the assessment or prediction of the course of the disease, as well as the prediction of the response to a treatment regimen.

The methods of the preset invention, which involve the analysis of blood samples, rely, inter alia, on VOCs linked to the incidence of Circulating Tumor Cells (CTCs). Although CTCs may serve as a biomarker of cancer with considerable clinical potential, there exist many obstacles before this potential can be realized (mainly rarity and viability after manipulation). Analyzing a VOCs pattern can bypass some of these obstacles since it does not rely on collecting the cells, but rather on their influence in relation to their environment. The principle of this approach is that CTC-related changes are reflected as measurable changes in the blood. Urine samples are typically used in urological cancers diagnosis. It has been surprisingly found that analyzing both blood and urine samples by the methods of the present invention results in higher accuracy of diagnosing even when non-urological cancers are involved, such as, e.g., lung cancer.

The present invention provides detection of cancer VOCs directly from a combination of human blood and urine samples. This approach exploits the routine tests of blood and urine done in regular cancer diagnosis procedure and cancer handling. Blood and urine analyses are simple minimally/non-invasive routine tests both in community medicine and hospitals, which regulations are well known and handled. Therefore, an additional analysis of these samples will be easy to integrate in common procedures. The present invention offers better accuracy, sensitivity and specificity values than hitherto known tests.

The present invention beneficially combines complementary information from different liquid biological samples. The combination of data obtained from these sources gives a wide picture of the patients' clinical state. Unlike breath analysis which shows strength in lung and upper gastric diseases, systemic approach provides more accurate information and improves sensitivity and specificity of diagnosing and monitoring such complex disease as cancer.

The present invention provides, inter alia, a method of diagnosing various types of cancers, which is based on a portable (also termed herein “mobile” device that can sense internal molecular alterations in the headspace of small samples of urine and blood, and in particular, volatile organic compounds released from or being present as a result of cancer cells incidence. The mobile device preferably should have miniaturized dimensions and comprise chemically sensitive nanosensors. The advantages of the system are that it is non-invasive, small, easy-to-use, inexpensive and is capable of obtaining and transmitting data in real time.

The present invention further provides a diagnostic method, which is based on the detection and measuring the levels of a predefined set of specific VOCs in the headspace of urine and blood samples, wherein the same set of VOCs allows to diagnose various types of cancer.

In the various aspects and embodiments, the present invention provides a method of diagnosing, monitoring, prognosing or differentiating between cancer selected from breast cancer, pancreatic cancer, ovarian cancer, lung cancer, gastric cancer, colon cancer, kidney cancer, head and neck cancer, prostate cancer, and combinations thereof in a test subject, comprising measuring and analyzing the levels of a set of volatile organic compounds (VOCs) in a blood sample and a urine sample obtained from the test subject.

The term “volatile organic compound (VOC)”, as used herein, is intended to encompass organic compounds having high or low volatility (such as semi-volatile organic compounds), inorganic volatile compounds (VCs), other solvents, volatile toxic chemicals, and volatile explosives.

The terms “VOCs in samples” and “VOCs in a test sample”, as used herein, refer to VOCs which are obtained from a headspace of the blood, urine, or body tissue sample.

The set of volatile organic compounds can be specific to a particular type of cancer. In such case, a plurality of different VOCs sets can be used in order to diagnose a particular type of cancer. Alternatively (and preferably), the set of volatile organic compounds can be a universal biomarker set, which allows to identify each one of the above-mentioned cancer types by using a single set of VOCs.

According to some embodiments, the set of volatile organic compounds comprises at least one of 2-nonen-1-ol, 2-ethyl-1-hexanol; (E)-2-decenal; octanediamide, N,N′-di-benzoyloxy-; (Z)-7-hexadecenal; benzene, 1,3-bis(1,1-dimethylethyl)-; 1,2-15,16-diepoxyhexadecane; tetradecane; and combinations thereof.

According to some embodiments, the set of volatile organic compounds comprises at least one volatile organic compound selected from the group consisting of 3-pentanone; toluene; 2,4-dimethylpentane; 1,2-dimethylbenzen; 4-methyloctane; cyclohexanone; 2-cyclohexen-1-one; 2-ethyl hexanal; 4-methyl nonane; phenol; alpha methyl styrene; benzene, 1,2,4-trimethyl-; 5-methyl-decane; tetramethyl butanedinitrile; 4-methyl-decane; 2-ethyl-1-hexanol; benzene methanol, alpha., alpha-dimethyl-; 2-butyl-1-octanol; nonanal; benzoic acid; 2-methyl dodecane; benzene, 1,3-bis(1,1-dimethylethyl)-; 1-chlorooctadecane; naphthalene, 1-methyl-; tetradecane; 1-dodecane; tetradecane, 2,6,10-trimethyl-; phenol, 2,6-bis(1,1-dimethylethyl)-4-methyl-; 2,2,4-trimethyl-1,3-pentanediol diisobutyrate; pentacosane; heptacosane; and combinations thereof.

According to some embodiments, the set of volatile organic compounds comprises at least one volatile organic compound selected from the group consisting of 2-propanol, 2-methyl-; 2-methyl furane; ethyl acetate; 2-methyl-1-propanol; 1-butanol; cyclobutane, ethenyl-/cyclopentene, 1-methyl-; cyclohexene; methyl methacrylate; butanoic acid, 3-hydroxy-, methyl ester, (S)-; 3-penten-1-ol; toluene; 3-ethyl-3-hexene; heptane, 2,4-dimethyl-; 2,4-dimethyl-1-heptene; 2-hexanol, 5-methyl-; cyclohexanol; 2-octen-1-ol, (E)-; 2,5-dihydroxybenzaldehyde, 2TMS derivative; hexanal, 2-ethyl-; heptane, 2,2,4,6,6-pentamethyl-; benzene, 1,3,5-trimethyl; decane, 4-methyl-; tetramethyl butanedinitrile; 1-hexanol, 2-ethyl; dodecane, 4,6-dimethyl-; benzenamine, N-methyl-; octan-2-one, 3,6-dimethyl-; 1-tetradecanol; dodecane, 2-methyl-; 2-octen-1-ol, 3,7-dimethyl-, isobutyrate, (Z)-; benzene, 1,3-bis(1,1-dimethylethyl)-; tetradecane, 2,6,10-trimethyl-; naphthalene, 1-methyl-; 1-tetradecanol; octadecane, 1-chloro-/2-hexadecen-1-ol, 3,7,11,15-tetramethyl-, [R—[R*,R*-(E)]]-; 1,3,5-triazine-2,4-diamine, 6-chloro-N-ethyl-; diphenyl ether; 1,6-dimethyl naphthalene; butylated hydroxytoluene; docosane, 11-decyl-; 2,2,4-trimethyl-1,3-pentanediol diisobutyrate; diethyl phthalate; tert-hexadecanethiol; docosane, 11-decyl-; 1,3-di-iso-propylnaphthalene; benzenesulfonamide, N-butyl-; hexadecanoic acid; octadecanoic acid; tetracosane; 1,2-benzenedicarboxylic acid, bis(2-ethylhexyl) ester, and combinations thereof.

According to some embodiments, the set of volatile organic compounds comprises at least one volatile organic compound selected from the group consisting of 2-propanol, 2-methyl; 2-butanone; acetic acid; chloroform; benzene; 1-butanol; 2-pentanone; isopentenyl alcohol (3-buten-1-ol, 3-methyl-); 1-pentanol; hexanal; chloro-benzene; ethyl-benzene; 1,3-dimethyl benzene; 2-heptanone; 1,2-dimethyl benzene/p-xylene/styrene; 2-methyl-cyclopentanone; benzaldehyde; 1-octen, 3-ol/phenol/carbamic acid, methyl-, phenyl ester; heptane, 2,2,4,6,6-pentamethyl-; 1,2,4-trimethylbenzene; 1,3,5-trimethylbenzene; 1,4-dichloro benzene; 2-ethyl 1-hexanol; benzene acetaldehyde; dodecane/2-butyl, 1-octanol; 2-octen, 1-ol (z); acetophenone; dodecane; undecane; nonanal; 2-[(trimethylsilyl)oxy]-2-{4-[(trimethylsilyl)oxy]phenyl}ethanamine; tridecane; decanal; benzothiazole; tetradecane, 1-chloro; 1-decene/cyclododecane; 1-octadecanesulphonyl chloride/1-chloro octadecane; 2-ethylbutyric acid, eicosyl ester; naphthalene, 1-methyl; 2,3-dichloro-benzeneamine; tricyclo[5.4.0.02,8]undec-9-ene, 2,6,6,9-tetramethyl-/(+)-aromadendrene/beta cedrane; 1H-3a,7-methanoazulene, 2,3,6,7,8,8a-hexahydro-1,4,9,9-tetramethyl-, (1.alpha.,3a.alpha.,7.alpha.,8a.beta.)-; 1H-cycloprop[e]azulene, 1a,2,3,4,4a,5,6,7b-octahydro-1,1,4,7-tetramethyl-, [1aR-(1a.alpha.,4.alpha.,4a.beta.,7b.alpha)]-; tetradecane; (8R,8aS)-2-Isopropylidene-8,8a-dimethyl-1,2,3,7,8,8a-hexahydronaphthalene; 4,4-dimethyl-3-(3-methylbut-3-enylidene)-2-methylenebicyclo[4.1.0]heptane; 1-tetradecanol; 5-hydroxymethyl-1,1,4a-trimethyl-6-methylenedecahydronaphthalen-2-ol/1-chloro hexadecane; 1-chloro octadecane/pentadecane/3,5,11-eudesmatriene/phenol, 2,4-bis(1,1-dimethylethyl)-; alpha-patchoulene; tetradecane, 2,6,10-trimethyl-/docosane, 11-decyl-; 1H-cycloprop[e]azulene, 1a,2,3,5,6,7,7a,7b-octahydro-1,1,4,7-tetramethyl-, [1aR-(1a.alpha.,7.alpha.,7a.beta.,7b.alpha)]-; 1,5,9 trimethyl cyclododecatriene; triazabicyclodecene (TBD); secobarbital; dotriacontane; [1,1′-Biphenyl]-2-ol, 5-(1,1-dimethylethyl)-; oleic acid; benzenesulfonamide, N-butyl-; heptacosane, and combinations thereof.

According to some embodiments, the set of volatile organic compounds comprises at least one volatile organic compound selected from the group consisting of hexane; 3-methyl butanal; pentanal; 2.3-dihydrofuran; hexanal; 1,3,5-trimethyl cyclohexane; 2,4-dimethyl1-heptene; 2,4-dimethyl decane; 4,7-dimethyl undecane; 2,4-dimethyl heptane; 4-methyl octane; 2-ethyl 1-hexanol; dodecane; 5-ethyl,2-methyl octane, and combinations thereof.

According to some embodiments, the set of VOCs comprises at least five VOCs selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, and 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, 3-methyl butanal. According to some embodiments, the set of VOCs comprises at least six VOCs from the above list, at least seven VOCs, at least eight VOCs, at least nine VOCs or at least ten VOCs. Each possibility represents a separate embodiment of the invention.

According to some embodiments, the set of VOCs comprises at least five VOCs selected from the group consisting of hexane, 3-methyl butanal, pentanal, 2.3-dihydro furan, hexanal, 1,3,5-trimethyl cyclohexane, 2,4-dimethyl1-heptene, 2,4-dimethyl decane, 4,7-dimethyl undecane, 2,4-dimethyl heptane, 4-methyl octane, 2-ethyl 1-hexanol, dodecane, 5-ethyl,2-methyl octane, and combinations thereof.

According to some embodiments, the set of VOCs comprises at least five VOCs selected from the group consisting of 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydro furan, 2,3,5 trimethyl hexane, 2-pentyl furan, and 3-methyl butanal.

According to certain embodiments, the set of VOCs comprises 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydrofuran, 2,3,5 trimethyl hexane, 2-pentyl furan, and 3-methyl butanal.

According to some embodiments, the set of VOCs comprises at least five VOCs selected from the group consisting of hexane, 3-methyl butanal, pentanal, 2.3-dihydro furan, hexanal, 1,3,5-trimethyl cyclohexane, 2,4-dimethyl1-heptene, 2,4-dimethyl decane, 4-methyl octane, and 5-ethyl,2-methyl octane.

According to some embodiments, the set of VOCs comprises at least five VOCs selected from the group consisting of 3-methyl butanal, pentanal, hexanal, 2,3-dihydro furan, 2,4-dimethyl decane, dodecane, 2-ethyl hexanol, 5-ethyl-2-methyl octane.

It should be understood that when measuring and analyzing both the blood and urine samples, not all the VOCs of the predetermined set of VOCs should be measured and analyzed in both the blood and urine samples—some VOCs may be measured and analyzed only in the blood sample, some VOCs may be measured and analyzed only in the urine sample and some VOCs may be measured and analyzed in both the blood sample and urine sample. In some embodiments, each one of the VOCs of the set of VOCs is measured and analyzed in both the blood sample and the urine sample. In some currently preferred embodiments, it is, however, prerequisite that at least one VOC from the at least five VOCs of the set of VOCs is measured and analyzed in the blood sample and at least one VOC from the at least five VOCs of the set of VOCs is measured and analyzed in the urine sample.

In some embodiments, the VOCs to be measured and analyzed in the blood sample are selected from the group consisting of 4-heptanone, dodecane, 2-heptanone, 2-heptanone, 2-methyl 2-propanol, heptanal, 1-octene-3-ol, 3-ethyl 3-methylheptane, tetradecane, 2,4 dimethyl decane, hexanal, pentanal, 2,3 dihydrofuran, 2,3,5 trimethyl hexane, 2-pentyl furan, and 3-methyl butanal.

In some embodiments, the VOCs to be measured and analyzed in the urine sample are selected from the group consisting of 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 2-heptanone, dodecane, 5-ethyl 2-methyl octane, 3-Hexanone, and 2,3,5 trimethyl hexane.

In some embodiments, the VOCs to be measured and analyzed in both the blood sample and the urine sample are dodecane and 2-heptanone.

The levels of volatile organic compounds in a sample can be measured by the use of at least one technique selected from Gas-Chromatography (GC), GC-lined Mass-Spectrometry (GC-MS), Proton Transfer Reaction Mass-Spectrometry (PTR-MS), Electronic nose device, and Quartz Crystal Microbalance (QCM). Gas-Chromatography-Mass Spectrometry can be combined with a thermal desorber or with in-tube Extraction (ITEX), In certain embodiments, measuring the levels of a set of VOCs comprises the use of Gas-Chromatography-Mass Spectrometry (GC-MS) combined with In-tube Extraction (ITEX).

The levels of the VOCs can be analyzed with a pattern recognition analyzer. According to some embodiments, the pattern recognition analyzer comprises at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

According to some embodiments, analyzing the set of VOCs is performed by using a model based on a database of levels of the set of VOCs in control samples comprising blood and urine samples. The control samples, according to the principles of the present invention are obtained from a control individual, i.e., an individual not having cancer (negative control) or an individual afflicted with a certain type of cancer (positive control), wherein cancer is selected from the group consisting of breast cancer, pancreatic cancer, ovarian cancer, lung cancer, gastric cancer, colon cancer, head and neck cancer and prostate cancer, or any other chronic disease, such as, but not limited to, fibro gastroscopy. Control samples obtained from the individual afflicted with a certain type of cancer can further include individuals having different stages of the same cancer. According to some embodiments, the model is developed by using at least one algorithm selected from the algorithms listed hereinabove. For example, the algorithm can be used to choose the VOCs which allow forming individual clusters for each type of a control sample, i.e., which would allow to distinguish between different types of cancer, different stages of cancer or between healthy and sick subjects.

A set of VOCs can further be determined by the distributions of VOCs in samples from cancer patients in comparison to the distributions of the same VOCs in control samples. The set of VOCs can comprise specific VOCs for which a statistically significant difference in their level in samples from cancer patients as compared to samples from control subjects exists. The term “significantly different” as used herein refers to a quantitative difference in the concentration or level of each VOC from the set or combinations of VOCs as compared to the levels of VOCs in control samples obtained from individuals not having cancer. A statistically significant difference can be determined by any test known to the person skilled in the art. Common tests for statistical significance include, among others, t-test, ANOVA1 Kruskal-Wallis, Wilcoxon, Mann-Whitney and odds ratio. Individual samples (of unknown status) can be compared with data from the reference group (negative control), and/or compared with data obtained from a positive control group known to have cancer. An increase or decrease in the level as compared to a control or reference value or mean control level or reference value, or a change, difference or deviation from a control or reference value, can be considered to exist if the level differs from the control level or reference value, by about 5% or more, by about 10% or more, by about 20% or more, or by about 50% or more compared to the control level or reference value. Statistical significance may alternatively be calculated as P<0.05. Methods of determining statistical significance are known and are readily used by a person of skill in the art. In a further alternative, increased levels, decreased levels, deviation, and changes can be determined by recourse to assay reference limits or reference intervals. These can be calculated from intuitive assessment or non-parametric methods. Overall, these methods calculate the 0.025, and 0.975 fractals as 0.025*(n+1) and 0.975*(n+1). Such methods are well known in the art. The presence of a VOC marker which is absent in a control sample, is also contemplated as an increased level, deviation or change. The absence of a VOC marker which is present in a control, for example, is also contemplated as a decreased level, deviation or change.

The set of volatile organic compounds which are indicative of cancer selected from the group consisting of breast cancer, pancreatic cancer, ovarian cancer, lung cancer, gastric cancer, colon cancer, kidney cancer, head and neck cancer, prostate cancer, and combinations thereof, can comprise VOCs that are present in blood and urine samples of cancer patients in levels which are at least one standard deviation [SD] larger or smaller than their mean level in respective samples of a negative control population. More preferably, the levels of VOCs in samples of cancer patients are at least 2[SD] or 3[SD] larger or smaller than their mean level in samples of a negative control population. Accordingly, individual samples (of unknown status) are considered to belong to a sick population when the level of VOCs is at least 1[SD], 2[SD] or 3[SD] larger or smaller than the mean level of VOCs in samples of a negative control population.

The difference between samples obtained from cancer patients and control samples for the identification of the specific VOCs set can further be assessed by the algorithms mentioned hereinabove.

When analyzing the VOCs of the VOCs set in a test sample, the identified VOCs levels can be compared with reference levels of said VOCs derived from a database of said VOCs detected in the urine and blood samples of subjects afflicted with a known type of cancer, wherein the combination of the reference levels of each of the VOCs of the VOCs set is characteristic of a particular cancer type, selected from the group consisting of breast cancer, pancreatic cancer, ovarian cancer, lung cancer, gastric cancer, colon cancer, head and neck cancer and prostate cancer. The method can further include selecting the closest match between the levels of said VOCs from the test sample and the combination of the reference levels of the VOCs of the VOCs set.

The terms “test subject” and “control subject” as used herein refer a mammal, preferably humans.

The diagnosis, prognosis and/or monitoring of cancer comprises the diagnosis of a subject who is at risk of developing cancer, a subject who is suspected of having cancer, or a subject who was diagnosed with cancer using commonly available diagnostic tests (e.g., computed tomography (CT) scan). The present invention further provides the monitoring of cancer in patients having cancer. The term “monitoring” as used herein refers to the monitoring of disease progression or disease regression following treatment. Also encompassed by this term is the evaluation of treatment efficacy using the methods of the present invention.

According to some embodiments, the cancer is selected from the group consisting of kidney cancer, gastric cancer, and lung cancer. Each possibility represents a separate embodiment of the invention.

According to some currently preferred embodiments, analyzing each one of the blood sample and the urine sample provides enhanced accuracy, sensitivity and/or specificity of the diagnosing as compared to analyzing only one of said blood sample and urine sample.

According to some embodiments, the method comprises analyzing levels of the set of VOCs in a body tissue sample obtained from the test subject.

In various aspects and embodiments, the present invention provides a method for diagnosing, monitoring, prognosing, or differentiating between cancer selected from breast cancer, pancreatic cancer, ovarian cancer, lung cancer, gastric cancer, colon cancer, head and neck cancer, kidney cancer, prostate cancer, and combinations thereof, or stages thereof, in a test subject, the method comprising exposing an array of chemically sensitive sensors comprising a material selected from the group consisting of conductive nanostructures coated with an organic coating, a conducting polymer and a conductive polymer composite, to a blood sample and/or a urine sample obtained from the test subject.

In some currently preferred embodiments, the method comprising exposing the array of the chemically sensitive sensors to both the blood sample and the urine sample.

In various aspects and embodiments, the present invention provides a method of diagnosing, monitoring, prognosing, or differentiating between cancer selected from breast cancer, pancreatic cancer, ovarian cancer, lung cancer, gastric cancer, colon cancer, head and neck cancer, kidney cancer, prostate cancer, and combinations thereof, or stages thereof, in a test subject, the method comprising contacting a portable device with a blood sample and/or a urine sample obtained from the test subject. The portable device comprises an array of chemically sensitive sensors comprising a material selected from the group consisting of conductive nanostructures coated with an organic coating, a conducting polymer and a conductive polymer composite.

In some currently preferred embodiments, the method comprising contacting the portable device with both the blood sample and the urine sample.

The conductive nanostructures can comprise conductive nanoparticles such as, e.g., metal and metal alloy nanoparticles. Non-limiting examples of suitable metals and metal alloys include Au, Ag, Ni, Co, Pt, Pd, Cu, Al, Au/Ag, Au/Cu, Au/Ag/Cu, Au/Pt, Au/Pd, Au/Ag/Cu/Pd, Pt/Rh, Ni/Co, and Pt/Ni/Fe.

The coating of the conductive nanoparticles can comprise a monolayer or multilayers of organic compounds, wherein the organic compounds can be small molecules, monomers, oligomers or polymers, preferably with short polymeric chains. Non-limiting examples of suitable organic compounds include alkylthiols, arylthiols, alkylarylthiols, alkylthiolates, ω-functionalized alkylthiolates, arenethiolates, (γ-mercaptopropyl)tri-methyloxysilane, dialkyl sulfides, diaryl sulfides, alkylaryl sulfides, dialkyl disulfides, diaryl disulfides, alkylaryl disulfides, alkyl sulfites, aryl sulfites, alkylaryl sulfites, alkyl sulfates, aryl sulfates, alkylaryl sulfates, xanthates, oligonucleotides, polynucleotides, dithiocarbamate, alkyl amines, aryl amines, diaryl amines, dialkyl amines, alkylaryl amines, arene amines, alkyl phosphines, aryl phosphines, dialkyl phosphines, diaryl phosphines, alkylaryl phosphines, phosphine oxides, alkyl carboxylates, aryl carboxylates, dialkyl carboxylates, diaryl carboxylates, alkylaryl carboxylates, cyanates, isocyanates, peptides, proteins, enzymes, polysaccharides, phospholipids, and combinations and derivatives thereof.

In an aspect and embodiments of the present invention, the thiol is selected from the group consisting of dodecanethiol, 4-tert methyl-benzenethiol, 2-ethylhexanethiol, decanethiol, 4-chlorobenzenemethanethiol, 3-ethoxytiophenol, hexanethiol, octadecanethiol, and combinations thereof.

According to a specific embodiment, alkylthiol or alkylarylthiol is selected from the group consisting of tert-dodecanethiol, butanethiol, 4-cholorobenzenemethanthiol, 4-tert butylbenzenethiol, 2-naphthalenethiol, 2-nitro-4-(trifluoromethyl)benzenethiol, dodecanethiol, decanethiol, octadecanethiol, 3-ethoxythiophenol, benzylmercaptan, hexanethiol, 2-ethylhexanethiol, 1,6-hexanedithiol, and combinations thereof.

Sensors comprising metal nanoparticles capped with various organic coatings can be synthesized as is known in the art, for example using the two-phase method (Brust et al., J. Chem. Soc. Chem. Commun., 1994, 801, 2) with some modifications (Hostetler et al., Langmuir, 1998, 14, 24). Capped gold nanoparticles can be synthesized by transferring AuCl₄ from aqueous HAuCl₄·xH2O solution to a toluene solution by the phase-transfer reagent TOAB. After isolating the organic phase, excess thiols are added to the solution. The mole ratio of thiol:HAuCl₄·xH2O can vary between 1:1 and 10:1, depending on the thiol used. This is performed in order to prepare mono-disperse solution of gold nanoparticles in average size of about 2-5 nm. After vigorous stirring of the solution, aqueous solution of reducing agent NaBH4 in large excess is added. The reaction is constantly stirred at room temperature for at least 3 hours to produce a dark brown solution of the thiol-capped Au nanoparticles. The resulting solution is further subjected to solvent removal in a rotary evaporator followed by multiple washings using ethanol and toluene. Gold nanoparticles (GNPs) capped with particular thiols, can be synthesized by ligand—exchange method from pre-prepared hexanethiol-capped gold nanoparticles. In a typical reaction, excess of thiol is added to a solution of hexanethiol-capped gold nanoparticles in toluene. The solution is kept under constant stirring for few days in order to allow as much ligand conversion as possible. The nanoparticles are purified from free thiol ligands by repeated extractions.

The synthesized coated gold nanoparticles can then be assembled (e.g. by a self-assembly process) to produce 1D wires or a film of capped nanoparticles. The term “film”, as used herein, corresponds to a configuration of well-arranged assembly of capped nanoparticles. 2D or 3D films of coated nanoparticles may be used. Exemplary methods for obtaining well-ordered two- or three-dimensional assemblies of coated nanoparticles include, but are not limited to,
i. Random deposition from solution of capped nanoparticles on solid surfaces. The deposition is performed by drop casting, spin coating, spray coating and other similar techniques. According to some embodiments, gold nanoparticles coated with decanethiol are drop-casted onto a sensor substrate. The sensor can further be dried at ambient conditions and/or at elevated temperature ranging from about 35° C. to about and reduced pressure (e.g., in a vacuum oven).
ii. Field-enhanced or molecular-interaction-induced deposition from solution of capped nanoparticles on solid surfaces.
iii. Langmuir-Blodgett or Langmuir-Schaefer techniques. The substrate is vertically plunged through self-organized/well-ordered 2D monolayer of capped nanoparticles at the air-subphase interface, wherein the latter is being subsequently transferred onto it. Multiple plunging of the substrate through the 2D monolayer of capped nanoparticles at the air-subphase interface results in the fabrication of the 3D-ordered multilayers of capped nanoparticles.
iv. Soft lithographic techniques, such as micro-contact printing (mCP), replica molding, micro-molding in capillaries (MIMIC), and micro-transfer molding (mTM). These methods are based on variations of self-assembly and replica molding of organic molecules and polymeric materials, for fabricating capped nanoparticles from nanometer-scale to a mesoscopic scale (Zhao et al., J. Mater. Chem., 1997, 7(7), 1069).
v. Various combinations of Langmuir-Blodgett or Langmuir-Schaefer methods with soft lithographic techniques can be used to produce patterned Langmuir-Blodgett films of molecularly modified capped nanoparticles which are transferred onto solid substrates.
vi. Printing on solid-state or flexible substrates using an inject printer designated for printed electronics. A solution containing the capped nanoparticles is used as a filling material (or “ink”) of the printing head according to procedures well known in the art.

The metal nanoparticles may have any desirable morphology including, but not limited to, a cubic, a spherical, and a spheroidal morphology. The mean particle size of the metal nanoparticles can range from about 1 to about 10 nm. The synthesized nanoparticles can be assembled (e.g., by a self-assembly process) to produce 1D wires or a film of capped nanoparticles.

According to some embodiments, the array comprises a material selected from gold nanoparticles (GNPs) with tert-dodecanethiol, GNPs with butanethiol, GNPs with 4-cholorobenzenemethanthiol, GNPs with 4-tert butylbenzenethiol, GNPs with 2-naphthalenethiol, GNPs with 2-nitro-4-(trifluoromethyl)benzenethiol, GNPs with dodecanethiol, GNPs with decanethiol, GNPs with octadecanethiol, GNPs with 3-ethoxythiophenol, GNPs with benzylmercaptan, GNPs with hexanethiol, GNPs with 2-ethylhexanethiol, and GNPs with 1,6-hexanedithiol.

In an aspect and embodiments of the present invention, the array comprises gold nanoparticles coated with octadecanethiol.

According to some embodiments, the array further comprises gold nanoparticles coated with an organic coating selected from the group consisting of tert-dodecanethiol, butanethiol, 4-cholorobenzenemethanthiol, 4-tert butylbenzenethiol, 2-naphthalenethiol, 2-nitro-4-(trifluoromethyl)benzenethiol, dodecanethiol, decanethiol, 3-ethoxythiophenol, benzylmercaptan, hexanethiol, 2-ethylhexanethiol, 1,6-hexanedithiol, butanethiol, dibutyl disulfide, and combinations thereof. In certain embodiments, the array further comprises gold nanoparticles coated with decanethiol and gold nanoparticles coated with 3-ethoxythiophenol. According to some exemplary embodiments, the array further comprises gold nanoparticles coated with decanethiol, gold nanoparticles coated with 3-ethoxythiophenol, gold nanoparticles coated with dodecanethiol, gold nanoparticles coated with 2-ethylhexanethiol, and gold nanoparticles coated with tert-dodecanethiol.

In certain embodiments, the array comprises gold nanoparticles coated with octadecanethiol, gold nanoparticles coated with decanethiol, gold nanoparticles coated with 3-ethoxythiophenol, gold nanoparticles coated with decanethiol, gold nanoparticles coated with 3-ethoxythiophenol, gold nanoparticles coated with dodecanethiol, gold nanoparticles coated with 2-ethylhexanethiol, and gold nanoparticles coated with tert-dodecanethiol.

The conductive nanostructures can comprise single-walled carbon nanotubes (SWCNTs).

The term “single walled carbon nanotube” as used herein refers to a cylindrically shaped thin sheet of carbon atoms having a wall which is essentially composed of a single layer of carbon atoms which are organized in a hexagonal crystalline structure with a graphitic type of bonding. A nanotube is characterized by the length-to-diameter ratio. It is to be understood that the term “nanotubes” as used herein refers to structures in the nanometer as well as micrometer range.

The single-walled carbon nanotubes can have diameters ranging from about 0.5 nanometers (nm) to about 100 nm and lengths ranging from about 50 nm to about 10 millimeters (mm). More preferably, the single-walled carbon nanotubes can have diameters ranging from about 0.7 nm to about 50 nm and lengths ranging from about 250 nm to about 1 mm. Even more preferably, the single-walled carbon nanotubes can have diameters ranging from about 0.8 nm to about 10 nm and lengths ranging from about 0.5 micrometer (μm) to about 100 μm. Most preferably, the single-walled carbon nanotubes can have diameters ranging from about 1 nm to about 2 nm and lengths ranging from about 1 μm to about 20 μm.

The nanotubes can be arranged in a random network configuration. In some embodiments, the network of SWCNTs can be fabricated by a physical manipulation or in a self-assembly process. The term “self-assembly”, as used herein, refers to a process of the organization of molecules without intervening from an outside source. The self-assembly process occurs in a solution/solvent or directly on a solid-state substrate.

Main approaches for the synthesis of carbon nanotubes in accordance with the present invention include, but are not limited to, laser ablation of carbon, electric arc discharge of graphite rod, and chemical vapor deposition (CVD) of hydrocarbons. Among these approaches, CVD coupled with photolithography has been found to be the most versatile in the preparation of various carbon nanotube devices. In a CVD method, a transition metal catalyst is deposited on a substrate (e.g. silicon wafer) in the desired pattern, which may be fashioned using photolithography followed by etching. The silicon wafer having the catalyst deposits is then placed in a furnace in the presence of a vapor-phase mixture of, for example, xylene and ferrocene. Carbon nanotubes typically grow on the catalyst deposits in a direction normal to the substrate surface. Various carbon nanotube materials are available from commercial sources.

Other CVD methods include the preparation of carbon nanotubes on silica (SiO₂) and silicon surfaces without using a transition metal catalyst. Accordingly, areas of silica are patterned on a silicon wafer, by photolithography and etching. Carbon nanotubes are then grown on the silica surfaces in a CVD or a plasma-enhanced CVD (PECVD) process. These methods provide the production of carbon nanotube bundles in various shapes.

The SWCNTs can be coated with polycyclic aromatic hydrocarbons (PAH) or derivatives thereof, such as hexa-peri-hexabenzocoronene (HBC) molecules. HBC molecules can be unsubstituted or substituted by any one of methyl ether (HBC-OC1), 2-ethyl-hexyl (HBC-C6,2), 2-hexyldecyl (HBC-C10,6), 2-decyltetradecyl (HBC-C14,10), and dodecyl (HBC-C12). In certain embodiments, the PAH is crystal hexakis(n-dodecyl)-peri-hexabenzocoronene (HBC-C12).

In some embodiments, the array comprises SWCNTs coated with PAH. In certain embodiments, the array comprises SWCNTs coated with HBC-C12.

The term “conducting polymer”, as used in some embodiments, refers to a polymer which is intrinsically electrically-conductive, and which does not require incorporation of electrically-conductive additives (e.g., carbon black, carbon nanotubes, metal flake, etc.) to support substantial conductivity of electronic charge carrier. In further embodiments, the term “conducting polymer” refers to a polymer which becomes electrically-conductive following doping with a dopant. In certain embodiments, said doping comprises protonation (also termed herein “protonic doping”). In still further embodiments, the term “conducting polymer” refers to a polymer which is electrically-conductive in the protonated state thereof, whether said protonation is either partial or full. Alternatively, conducting polymers can be doped via a redox reaction. In yet further embodiments, the term “conducting polymer” refers to a polymer which is electrically-conductive in the oxidized and/or reduced state thereof. The term “conducting polymer”, as used herein, refers in some embodiments to a semiconducting polymer. The term “semiconducting polymer”, as used in some embodiments, refers to a polymer which is intrinsically semi-conductive, and which does not require doping with charge transporting or withdrawing molecules or components to support substantial conductivity of electronic charge carrier. The conducting polymers suitable for use in the devices and methods of the present invention can have conductivity ranging from about 0.1 S·cm⁻¹ to about 500 S·cm⁻¹, from about 0.1 S·cm⁻¹ to about 100 S·cm⁻¹, or from about 0.1 S·cm⁻¹ to about 10 S·cm⁻¹.

Non-limiting examples of suitable conducting polymers include diketopyrrolopyrrole-naphthalene copolymer (PDPP-TNT), polydiketopyrrolopyrrole, polyaniline (PANI), polythiophene, poly(3,4-ethylenedioxythiophene)-poly(styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof.

According to some embodiments, the array further comprises the conducting polymer selected from the above list. In certain embodiments, said conducting polymer is diketopyrrolopyrrole-naphthalene.

The term “conductive polymer composite”, as used in some embodiments, refers to a combination of a polymer which is not intrinsically conductive with electrically-conductive additives (e.g., carbon black, carbon nanotubes, metal flake, etc.).

Non-limiting examples of the conductive polymer composite include a disulfide polymer, a methacrylate polymer, and/or a polyethyleneimine polymer mixed with a carbon powder, e.g., carbon black or graphite. Non-limiting examples of carbon black suitable for use in the conductive polymer composites include acetylene black, channel black, furnace black, lamp black and thermal black. The disulfide polymer can be a self-healing polymer.

According to some embodiments, the array further comprises the conductive polymer composite selected from the group consisting of a disulfide polymer, a methacrylate polymer, a polyethyleneimine polymer, combinations and derivatives thereof, wherein said polymer is mixed with a carbon powder. In certain embodiments, the array further comprises carbon black/poly(propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

According to some embodiments, the array comprises a material selected from random networks (RNs) of carbon nanotubes (CNTs) with crystal hexa-perihexabenzocoronene (HBC) with C12 (HBC-C12), carbon black (CB)/poly(propylene-urethaneureaphenyl-disulfide) (PPUU-2S) composite, CB/(poly (urethane-carboxyphenyl-disulfide) (PUC-2S)/PPUU-2S) composite, CB/(poly(propyl methacrylate) (PPMA)/polyethyleneimine (PEI)) composite, bilayers of single-wall carbon nanotubes and semi-triangular ester (methyl) polycyclic aromatic hydrocarbons (PAHs) (PAH-3), hexyldecyl-substituted poly(diketopyrrolopyrrole), pyrrolopyrrolediketopyrrolopyrrole-naphthalene (PDPP-TNT), diketopyrrolopyrrole-anthracene (PDPP-FAF), and combinations thereof.

According to some embodiments, the array comprises 2-naphthalenethiol GNPs, dodecanethiol GNPs, and decanethiol GNPs. According to some embodiments, the array comprises decanethiol GNPs and 2-naphthalenethiol GNPs. According to some embodiments, the array comprises tert-dodecanethiol GNPs. According to some embodiments, the array comprises tert-dodecanethiol GNPs, diketopyrrolopyrrole-anthracene (FAF), and 2-naphthalenethiol GNPs. According to some embodiments, the array comprises hexyldecyl-substituted poly(diketopyrrolopyrrole) and CB/(PUC-2S/PPUU-2S) composite. According to some embodiments, the array comprises decanethiol GNPs, CB/(PPMA/PEI) composite, and 3-ethoxythiophenol GNPs. According to some embodiments, the array comprises diketopyrrolopyrrole-naphthalene (TNT), decanethiol GNPs, and diketopyrrolopyrrole-benzothiadiazole (TBT).

According to some embodiments, the array comprises diketopyrrolopyrrole-anthracene (FAF) and 4-tert-butylbenzenethiol GNPs. According to some embodiments, the array comprises tert-dodecanethiol GNPs and diketopyrrolopyrrole-anthracene (FAF). According to some embodiments, the array comprises octadecanethiol GNPs and decanethiol GNPs. According to some embodiments, the array comprises tert-dodecanethiol GNPs and 1,6-hexanedithiol GNPs. According to some embodiments, the array comprises diketopyrrolopyrrole-naphthalene (TNT). According to some embodiments, the array comprises tert-dodecanethiol GNPs and dodecanethiol GNPs. According to some embodiments, the array comprises decanethiol GNPs and CB/(PPMA/PEI) composite.

According to some embodiments, the array comprises dodecanethiol GNPs and 2-ethylhexanethiol GNPs. According to some embodiments, the array comprises dodecanethiol GNPs and 3-ethoxytiophenol GNPs. According to some embodiments, the array comprises dodecanethiol GNPs, 2-ethylhexanethiol GNPs and 3-ethoxytiophenol GNPs. According to some embodiments, the array comprises 3-ethoxytiophenol GNPs. According to some embodiments, the array comprises 4-tert methyl-benzenethiol GNPs and 3-ethoxytiophenol GNPs. According to some embodiments, the array comprises 2-ethylhexanethiol GNPs, decanethiol GNPs, 3-ethoxytiophenol GNPs, and 4-chlorobenzene-methanethiol GNPs.

According to some embodiments, the array comprises dodecanethiol GNPs, 2-ethylhexanethiol GNPs, and 4-tert methyl-benzenethiol GNPs. According to some embodiments, the array comprises 4-tert methyl-benzenethiol GNPs, decanethiol GNPs, 3-ethoxytiophenol GNPs, and 4-chlorobenzene-methanethiol GNPs. According to some embodiments, the array comprises dodecanethiol GNPs and 3-ethoxytiophenol GNPs. According to some embodiments, the array comprises hexanethiol GNPs. According to some embodiments, the array comprises dodecanethiol GNPs and 3-ethoxytiophenol GNPs. According to some embodiments, the array comprises 2-ethylhexanethiol GNPs, decanethiol GNPs, and 3-ethoxytiophenol GNPs. According to some embodiments, the array comprises 3-ethoxytiophenol GNPs and 4-chlorobenzene-methanethiol GNPs.

According to some embodiments, the array comprises a material selected from tert-dodecanethiol GNPs; butanethiol GNPs; 4-chlorobenzenemethanethiol GNPs, 4-tert-butylbenzenethiol GNPs; dibutyl disulfide GNPs; 2-nitro-4-(trifluoromethyl)benzenethiol GNPs, octadecanethiol GNPs; decanethiol GNPs; 2-ethylhexanethiol GNPs tert-dodecanethiol GNPs; 3-ethoxythiophenol GNPs; benzylmercaptan GNPs; hexanethiol GNPs; diketopyrrolopyrrole-naphthalene; SWCNTs coated with Polycyclic Aromatic Hydrocarbon 3 (PAH-3); SWCNTs coated with HBC-C12; black carbon with poly(propylene-urethaneureaphenyl-disulfide) PPUU-2S; CB/(PUC-2S/PPUU-2S) composite—black carbon with poly(propylene-urethaneureaphenyl-disulfide) PPUU-2S mixed with poly(urethane-carboxyphenyl-disulfide) PUC-2S; and CB/(PPMA/PEI) composite—black carbon/poly(2-hydroxypropyl methacrylate) mixed with polyethyleneimine).

According to some embodiments, the array comprises tert-dodecanethiol GNPs; butanethiol GNPs; 4-chlorobenzenemethanethiol GNPs, 4-tert-butylbenzenethiol GNPs; dibutyl disulfide GNPs; 2-nitro-4-(trifluoromethyl)benzenethiol GNPs, octadecanethiol GNPs; decanethiol GNPs; 2-ethylhexanethiol GNPs tert-dodecanethiol GNPs; 3-ethoxythiophenol GNPs; benzylmercaptan GNPs; hexanethiol GNPs; polymer coated 2D random networks of single-walled carbon nanotubes; diketopyrrolopyrrole-naphthalene; SWCNTs coated with PAH-3; SWCNTs coated with HBC-C12; black carbon with poly(propylene-urethaneureaphenyl-disulfide) PPUU-2S; CB/(PUC-2S/PPUU-2S) composite—black carbon with poly(propylene-urethaneureaphenyl-disulfide) PPUU-2S mixed with poly(urethane-carboxyphenyl-disulfide) PUC-2S; and CB/(PPMA/PEI) composite—black carbon/poly(2-hydroxypropyl methacrylate) mixed with polyethyleneimine).

In some embodiments, the portable device comprises the array of the chemically conductive sensors comprising a material selected from the group consisting of gold nanoparticles coated with dodecanethiol, 4-tert methyl-benzenethiol, 2-ethylhexanethiol, decanethiol, 4-chlorobenzenemethanethiol, 3-ethoxytiophenol, hexanethiol, and octadecanethiol.

In some exemplary embodiments, the portable device comprises the array of the chemically conductive sensors comprising eight chemically sensitive nanosensors comprising gold nanoparticles coated with dodecanethiol, 4-tert methyl-benzenethiol, 2-ethylhexanethiol, decanethiol, 4-chlorobenzenemethanethiol, 3-ethoxytiophenol, hexanethiol, and octadecanethiol.

The chemically sensitive sensor typically further includes a set of electrodes, such as two, three or more electrodes or an electrode array, being in electric contact with the material of the sensor. In some embodiments, the sensor material is applied onto the electrodes. In some related embodiments, the sensor material forms a conductive path between the electrodes. The electrodes, which are coupled to the sensing material and/or are disposed beneath the sensing material can enable the measurement and transmittance of the electric signals generated by chemically sensitive sensor. The electrodes can be further used to apply a constant current or potential to the sensor.

In some embodiments, the sensor comprises an electrode array. The electrode array can include a pair of electrodes (a positive electrode and a negative electrode) or a plurality of said pairs of electrodes. The electrode array can further comprise patterned electrodes, for example, interdigitated electrodes.

The electrodes can comprise any metal having high conductivity. Non-limiting examples of suitable metals include Au, Ti, Cu, Ag, Pd, Pt, Ni, and Al.

In some embodiments, the sensor material and/or the electrodes are confined by a micro-barrier. Chemically sensitive sensors comprising a metal nanoparticles-based sensing layer, which is confined by the micro-barrier, provide highly uniform responses when exposed to VOCs. Additional information on the micro-barrier can be found in WO2020089901, which content is incorporated herein by references in its entirety.

The sensor material and/or the electrodes can be supported on a substrate, which can be rigid or flexible. Non-limiting examples of suitable substrates include paper, polymer, silicon, silicon dioxide, silicon rubber, ceramic material, metal, insulator, semiconductor, semimetals and combinations thereof. The polymer can be selected from polytetrafluoroethylene, polyamide, polyimide, polyester, polyimine, polyethylene, polyethylene terephthalate, polyvinyl chloride (PVC), polydimethylsiloxane, polystyrene, and derivatives and combinations thereof.

The chemically sensitive sensor can be configured, e.g., as a capacitive sensor, resistive sensor, chemiresistive sensor, impedance sensor, field effect transistor sensor, strain gauge sensor or the like.

The array of the chemically sensitive sensors can include a modified membrane for liquids. In some embodiments, said membrane is hydrophobic. The membrane can protect the material of the chemically sensitive sensors from aqueous liquid and/or gas found in the test sample. Non-limiting examples of a suitable material for the membrane include polyether sulfone (PES); polytetrafluoroethylene (PTFE); polypropylene (PP); cellulose acetate (CA); polyvinylidene fluoride (PVDF); polycarbonate (PC). The material of the membrane can be modified, for example, by negatively charged surface groups (e.g. sulfonic acid and carboxylic acid), increasing the hydrophilicity (including, inter alia, O₂/CO₂/N₂ plasma treatment, polyvinyl acetate, or phospholipids), introduction of steric hindrance (including, inter alia, by polysulfobetaine or polycarboxybetaine), biomimetic modification (including, inter alia, PEG, PEO, chitosan, or heparin) or asymmetric modification. Different methods can be used for the fabrication of the modified membrane, including chemical modification, copolymerization/blending, sputtering, Langmuir Blodgett, Atomic Layer Deposition, Atomic Vapor Deposition, Chemical Vapor Deposition and others.

The array of the chemically sensitive sensors is exposed to the blood sample and the urine sample individually. The blood sample and the urine sample obtained from the test subject can be disposed in separate headspace glass vials. In some embodiments, the vials remain closed for about 10 min to 5 hours before sampling. In further embodiments, the vials are heated to about 50° C. to 100° C. before sampling. The vials can be heated for about 5 min to about 60 min.

According to some embodiments, the step of exposing the array of the chemically sensitive sensors to the blood sample and/or the urine sample comprises subjecting the blood sample and the urine sample to pre-concentration. In some embodiments, the pre-concentration is performed on a Tenax TA tube.

The methods according to the principles of the present invention can further include a step measuring the output signals of the chemically sensitive sensors upon exposure of the array to the blood sample and/or urine sample. The signal of the sensors can be detected and/or measured by a suitable detection device, which is susceptible to a change in any one or more of resistance, conductance, alternating current (AC), frequency, capacitance, impedance, inductance, mobility, electrical potential, piezoelectricity, and voltage threshold.

The array of the chemically sensitive sensors can be communicatively coupled to the measuring device or electronic circuitry. In some embodiments, the array is electronically coupled to the measuring device or electronic circuitry.

According to further embodiments, the portable device further comprises a measuring device or electronic circuitry configured to measure the output signal of the array of the chemically sensitive sensors.

The method according to the principles of the present invention can further include analyzing output signals of the chemically sensitive sensors upon exposure of the array to the blood sample and/or urine sample.

When a plurality of sensors is used, the signals obtained from the sensors can be analyzed by a computing system configured for executing various algorithms stored on a non-transitory memory. Thus, according to some embodiments, the chemically sensitive sensor or sensor array is coupled to said computing system. The algorithms can be the same algorithms used for analyzing VOCs, as detailed hereinabove.

According to various aspects and embodiments of the invention, analyzing comprises using a model based on a database of response patterns of the array of the chemically sensitive sensors to control samples comprising blood and/or urine samples obtained from patients having the cancer and healthy subjects. According to the currently preferred embodiments, analyzing comprises using a model based on a database of response patterns of the array of the chemically sensitive sensors to control samples comprising both blood samples and urine samples obtained from patients having the cancer and healthy subjects. According to some embodiments, the model is developed based on an algorithm selected from the list of algorithms used for analyzing VOCs, as described hereinabove. For example, the algorithm can be used to train the array of chemically sensitive sensors for forming individual clusters of responses for each type of a control sample upon exposure thereto, which would allow to distinguish between different types of cancer, different stages of cancer or between healthy and sick subjects by using said array of chemically sensitive sensors.

According to some embodiments, analyzing the output signals comprises comparing said signals to a disease-specific pattern derived from said model, wherein each of the disease-specific patterns is characteristic of a particular cancer type, selected from the group consisting of breast cancer, pancreatic cancer, ovarian cancer, lung cancer, gastric cancer, colon cancer, head and neck cancer and prostate cancer. The method can further include selecting the closest match between the output signals of the at least one sensor and the database-derived disease-specific patterns.

According to some embodiments, analyzing the output signal of the chemically sensitive sensors comprises extracting a plurality of response-induced parameters from said signal, the response-induced parameters being selected from the group consisting of full non-steady state response at the beginning of the signal, full non-steady state response at the beginning of the signal normalized to baseline, full non-steady state response at the middle of the signal, full non-steady state response at the middle of the signal normalized to baseline, full steady state response, full steady state response normalized to baseline, area under non-steady state response, area under steady state response, the gradient of the response upon exposure of the at least one sensor, and the time required to reach 10%, 20%, 30%, 40%, 50%, 60%, 70%, 80%, 90% or 100% of the response upon exposure of the at least one sensor. Each possibility represents a separate embodiment of the invention.

It should be understood that not every chemically sensitive sensor in the array is responsive to cancer biomarker VOCs of both blood and urine samples. Some sensors in the array are responsive to cancer biomarker VOCs found in the blood sample, some sensors in the array are responsive to cancer biomarker VOCs found in the urine sample, and some sensors in the array are responsive to cancer biomarker VOCs found in the blood sample and to cancer biomarker VOCs found in the urine sample. However, exposure of each chemically sensitive sensor of the array to both blood and urine samples enhances the accuracy and sensitivity of the cancer diagnosis.

According to some embodiments, the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl Isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal.

In the embodiments related to the use of the portable device for diagnosing cancer in a test subject, the method comprises contacting the portable device with the blood sample and/or the urine sample obtained from the test subject. Said contacting can comprise drawing an aliquot of a headspace of the blood sample and/or an aliquot of a headspace of the urine sample into the device and exposing the array to said aliquot individually. The blood sample and the urine sample obtained from the test subject can be disposed in separate headspace glass vials.

Said aliquot of a headspace of the blood sample can have a volume ranging from about 0.5 μl to about 5 ml. In some embodiments, said aliquot of a headspace of the blood sample has a volume ranging from about 1 μl to about 1 ml.

Said aliquot of a headspace of the urine sample can have a volume ranging from about 0.5 μl to about 5 ml. In some embodiments, said aliquot of a headspace of the urine sample has a volume ranging from about 1 μl to about 1 ml.

The array of the chemically sensitive sensors can be sealed within the portable device from external atmosphere. In such manner, the array can be exposed to the aliquot of the headspace of the blood sample and/or the aliquot of the headspace of the urine sample, without diluting the sample and reducing the concentration of the cancer biomarker VOCs present in the sample headspace.

According to some embodiments, the portable device further comprises a sample inlet, a cannula, and a pipe, wherein the pipe is connected to the cannula at one end and to the sample inlet at another end. The cannula is configured to be inserted into the vial holding the blood sample and the vial holding the urine sample.

According to some embodiments, drawing an aliquot of a headspace of the blood sample and/or an aliquot of a headspace of the urine sample comprises inserting the cannula into a vial comprising the respective sample and pumping the headspace into the portable device. The pumping can be performed for a period ranging from about 0.5 s to about 60 s. In some embodiments, the period ranges from about 0.5 s to about 30 s, from about 0.5 s to about 20 s, from about 0.5 s to about 10, or from about 0.5 s to about 5 s. Each possibility represents a separate embodiment of the invention. In some embodiments, the pumping is performed for from about 0.5 s to about 2 s. In some exemplary embodiments, the pumping is performed for about 1 s.

The pumping rate can range from about 30 μl/s to about 3300 μl/s. In some embodiments, the pumping rate ranges from about 100 μl/s to about 1000 μl/s.

According to some embodiments, the canula is inserted into the vial up to about 0.5 cm above the liquid sample level. According to further embodiments, the canula is inserted into the vial up to about 1 cm above the liquid sample level.

In certain embodiments, the array is exposed to the aliquot of the headspace for a period ranging from about 1 s to about 5 min. In further embodiments, the array is exposed to each aliquot for a period ranging from about 5 s to about 120 s, from about 5 s to about 60 s, or from about 10 s to about 20 s. Each possibility represents a separate embodiment of the invention. In some embodiments, the array is exposed to each aliquot for a period of about 13 s.

According to some embodiments, the method further comprises drawing air from outside of the portable device (i.e., the external atmosphere, which is not the sample headspace) into the device. Said step can be performed after the step of drawing the aliquots of headspace of the samples and/or after the step of drawing the aliquots of headspace of the samples. According to some embodiments, said step is performed for a period ranging from about 0.5 s to about 300 s. The array can be exposed to the air for a period ranging from about 5 s to about 5 min.

According to some embodiments, the method further comprises drawing air from outside the portable device into the device for a period ranging from about 1 s to about 60 s and exposing the array thereto for from about 5 s to about 120 s.

According to some embodiments, the method involving the use of the portable device does not require preconcentration of the blood sample and/or the urine sample.

The array of the chemically sensitive sensors of the portable device allows analyzing extremely low concentrations of the cancer biomarker VOCs to provide reliable cancer diagnosis when using both the blood sample and the urine sample. The portable device can be conveniently used in hospitals during everyday procedures or in a doctor's office, providing real-time cancer diagnosis. The term “real-time”, as used herein, refers to a time period of up to about an hour between obtaining the blood sample and the urine sample from the patient and transmitting the diagnostic outcome of the analyzing step. The diagnostic outcome can be transmitted, e.g., to a mobile device or a remote server.

In some embodiments the portable device further comprises a transmitter. In some embodiments the array of the chemically sensitive sensors is configured to transmit an output signal to the transmitter upon exposure to the blood sample and the urine sample.

In some embodiments, the transmitter is communicatively coupled to the array and/or to a measuring device. In some embodiments, the transmitter is electronically coupled to the sensing unit and/or to a measuring device.

The transmitter can include a communication component for remote communication, as known in the art, including, inter alia, GSM/UMTS mobile broadband modem, Bluetooth, wireless data transmitter including Wi-Fi and communications satellites. Each possibility represents a separate embodiment of the invention.

In some embodiments, the transmitter receives an output signal of the array and transmits said signal to a remote server and/or to a portable electronic device. The remote server can comprise an algorithm, which analyzes said signal. The transmitter can further transmit the diagnosis outcome of the analyzing step to the portable electronic device. Non-limiting examples of the suitable portable electronic devices include a smartphone, a tablet, and a Chromebook.

According to some embodiments, the cancer is selected from the group consisting of kidney cancer, gastric cancer, and lung cancer. In certain embodiments, said cancer is kidney cancer. In certain embodiments, said cancer is gastric cancer. In certain embodiments, said cancer is lung cancer.

The methods according to various embodiments of the present invention can further include analysis of a body tissue sample, in addition to the blood and urine samples. The body tissue sample can be obtained, inter alia, from a biopsy. The handling of the body tissue sample in connection with the methods of the present invention, are similar to the handling of the blood and urine sample, wherein the tissue sample is placed into a vial and the array of the chemically sensitive sensors is exposed to the sample headspace.

Using cancerous body tissue brings an additional advantage. VOCs released by cancer cells and tissues can be measured while reducing contamination from other metabolic processes. In addition, the analysis of VOCs is much faster, inexpensive and does not necessitate qualified workers as histopathology of biopsy does. Moreover, unlike histopathology, VOC pattern from cancerous tissue can provide a wide spectrum of data including, but not limited to, genetic mutation background and altered biochemical pathways. The present invention allows testing tissue in-vivo in real time without the need of biopsy.

The diagnosing methods according to various aspects and embodiments of the invention can further comprise a step of treating the test subject if cancer is diagnosed. In some embodiments, treating the test subject at least one of a surgery, radiation therapy, chemotherapy, surveillance, adjuvant (additional) therapy, and targeted therapy.

As used herein and in the appended claims the singular forms “a”, “an,” and “the” include plural references unless the content clearly dictates otherwise. Thus, for example, reference to “a sample” includes a plurality of such samples and so forth. It should be noted that the term “and” or the term “or” are generally employed in its sense including “and/or” unless the content clearly dictates otherwise.

As used herein, the term “about” refers to a range of values ±20%, or ±10%, or ±5% of a specified value.

The principles of the present invention are demonstrated by means of the following non-limitative examples.

### EXAMPLES

### Example 1—Diagnosing Cancer by Analyzing Blood and Urine Samples Study Design

Study design: In total 304 patients with written consent were included in this study—130 control, 26 gastric cancer, 101 renal (kidney) cancer, 26 non-small lung cancer and 22 samples from dyspeptic patients who underwent fibrogastroscopy. The control samples were collected as part of an ongoing GISTAR population study (Full protocol in Leja. M, Park J Y, Murillo R, et al. Multicentric randomised study of Helicobacter pylori eradication and pepsinogen testing for prevention of gastric cancer mortality: the GISTAR study. BMJ Open. 2017 Aug. 11) where respondents aged 40-64 are invited from general population through family doctors if they have no known serious illnesses, such as cancer. They are then asked to fill out a supervised questionnaire, which cover extensive socio-economic factors, lifestyle and nutritional habits, medical history and phenotype data. All study participants were required to donate blood, stool, breath and, in the case of this study, urine. Samples were also collected from respondents with dyspeptic symptoms who were referred to and underwent fibrogastroscopy, in order to obtain a group of patients who display symptoms similar to gastric cancer. The samples from renal and gastric cancer patients were collected, in accordance to the inclusion criteria, in the Oncology Centre of Latvia while the non-small cell lung cancer samples were collected in the Centre of Tuberculosis and Lung Diseases, Riga. East Clinical University Hospital. All patients had their diagnosis confirmed pathologically and underwent a survey covering various lifestyle and socio-economic factors. Inclusion criteria for cancer groups: patients being referred for surgery or being investigated with the diagnosis of ‘gastric cancer’, ‘kidney cancer’ or ‘lung cancer’, recruitment should be prior to surgery, different cancer stages were enrolled. Exclusion criteria for cancer groups: age<18 years, patients with any other prevalent cancer of cancer diagnosis within the last 5-year period. The clinical trials received ethical approvals by the Ethical Committees of the respective hospital. In order to control for variations in time of freezing and shipping, lyophilized human plasma standards were frozen (Sigma, P9523-5ML) in multiple time-points. In total six standards were used by rehydrating with 5 ml of distilled, transferring to the same vacutainers used in this study and stored at −80° C. until shipping. Standards did not change significantly during freezing, shipping and defrosting and sampling.

### Example 2—Diagnosing Cancer by Analyzing VOCs in Blood and Urine Samples by GC-MS

In-tube extraction-Gas Chromatography-Mass Spectrometry (ITEX-GC-MS): GC-MS combined with In-tube extraction device (ITEX) was used for headspace sampling of human blood and urine samples. The sample vial was set on an automatic sampling system connected to the GC-MS (Auto-PAL-RSI 120). Automated ITEX applied a 1.3 mL headspace syringe with a Tenax TA-filled needle body. The VOCs were extracted from sample headspace by dynamic extraction on to the absorbent. The needle body was surrounded by a heating unit, which is used for VOCs desorption into the injection port of a GC-MS. The auto-sampler was equipped with a single magnet mixer (SMM) and a temperature-controlled tray holder. The samples were placed in the tray cooler at 25° C.; after transfer to the SMM, the sample was heated (70° C.) and stirred at 500 rpm for 60 min. The extraction volume of the gas phase was set to 1000 μL and 300 extraction strokes were used for the optimized method for each sample. The extraction flow-rate during extraction was set at 100 μL/sec. for desorption the ITEX trap was heated to 250° C. with desorption flow rate of 10 μL/sec into the hot injector. After desorption, the ITEX device was flushed with nitrogen gas at 260° C. for 5 min. The whole process (including injection, trap cleaning, and extraction of the following sample) was completed within the runtime of the GC oven program with cooling about 5h. An internal standard mixture (EPA-524) 1,4-Dichloro benzene-D4 was added (20 ppb) along with test samples as well as control plasma to ensure that the GC-MS was functioning effectively. Analysis of the compounds used a GC-7890B/MS-5977A instrument (Agilent). GC-MS was equipped with a SLB-5 ms capillary column (30 m length; 0.25 mm internal diameter; 1 μm thickness; Sigma-Aldrich), combined with a ITEX system. The samples were injected automatically from the ITEX into the GC-system. The following oven temperature profile was set: (a) 2 min at 50° C.; (b) 20° C./min ramp until 300° C.; and (c) 5 min at 300° C.

### Example 3—Diagnosing Cancer by Analyzing VOCs in Blood and Urine Samples by GC-MS-Data Analysis

GC-MS data processing: The GC-MS chromatograms were analyzed using Mass Hunter qualitative (version B.07.00; Agilent Technologies, USA) analysis. The compounds were tentatively identified through spectral library match NISTL.14 (National Institute of Standards and Technology, USA). To identify significant differences in VOCs between the groups, the Kruskal-Wallis test and an extension of the Non-parametric Wilcoxon test, including Bonferroni alpha correction were used. Hierarchical clustering using Ward's minimum variance method was applied including constellation plots. SAS JMP, Version. 14.0 (SAS Institute, Cary, North Carolina, USA; 1989, 2005) was used for statistical analysis.

Machine Learning: Three models have been developed, one using the blood data, a second one using the urine data. Finally, the last model combined the features from blood and urine data. In order to fairly compare all the models, only the patients with the combined data are used for the classification task (n=255). As the database contained low number of patients (n=255), a nested cross fold validation was used. This means that 5-fold cross-validation was performed five times, each time by rotating the train-test split (80% train, 20% test). Then the median and standard deviation of the metric on the test sets were reported. For each training set, a Bayesian optimization search over the hyper-parameters was performed, to find the best parameters. The metric used for optimization is F1 score. For each model, a Random Forest (RF) has been trained. The three models were trained and evaluated on the same train/test split. A total of three tasks have been conducted:
First task: binary classification task has been conducted, to diagnose whether a patient has cancer or not.
Second task: A multi-class classification for patients with cancer, to diagnose the type of cancer: kidney, lung or gastric.
Third task: A binary classification to distinguish between the 2 types of healthy patients: control and FG.

Data was standardized by subtracting the median and dividing by standard deviation: x_{new}=(x−median)/std. There is no missing data. Feature selection is applied on each fold. A Kruskal-Wallis test has been run for each feature, and if pᵥₐₗᵤₑ>threshold, the feature is removed. threshold is a hyper-parameter of the algorithm, and in this case threshold=0.05 was used.

For each model, the following metrics were reported:

Sensitivity(Se)=TP/(TP+FN),

Specificity(Sp)=TN/(FP+TN),

F1=2*TP/(2*TP+FP+FN),
where TP, TN, FP, FN are the True Positives, True Negatives, False Positives and False Negatives, respectively. The area under the ROC curve, AU ROC, was computed. For the multiclass classification task, the F1 score was computed using a micro average.

### Example 4 Diagnosing Cancer by Analyzing VOCs in Blood and Urine Samples by GC-MS—Results

The present experiment was designed to show that volatile organic compounds pattern can be used to detect cancer patients and distinguish them from non-cancer ones when analyzing blood and urine samples. For that, the headspace of blood and urine samples was analyzed by means of GC-MS and then both hierarchical clustering and random forest analysis were used to statistically assess the results to obtain highly accurate models for discriminating between the different study groups. The performance of a combined model based on pooled data obtained from both blood and urine samples was compared to a model based solely on the data obtained from urine samples and to a model based solely on the data obtained from blood samples.

The GC-MS has identified more than 100 VOCs in the different samples, but only 32 VOCs from each bio-fluid were selected for further investigation, a total of 64 (Table 1). For hierarchical clustering only 29 VOCs from blood and 22 VOCs from urine were used, these VOCs showed a significant difference between at least one-model comparisons. While for random forest analysis blood model, 26.8±1.0 (mean±std) features (i.e., VOCs) have been selected. For the urine model, 17.2±1.7. Finally, for the combined model, 41.4±2.1 features have been selected, including 26.2±1:0 features from blood data, and 15.2±1.1 features from urine data. The VOCs selected for the combined model (i.e., both blood sample and urine sample) were: 2,3,5,8-tetramethyl Decane (urine), Unknown (7) (blood), 3-Hexanone (urine), p-Cresol (urine), Unknown (17) (urine), Pentadecane (urine), 4,5 dimethyl Nonane (urine), Hexane (urine), 2,6 dimethyl Nonane (urine), 2-Nonanone (urine), 1-(3-methyl phenyl) Ethanone (urine), 3-Phenyl 2-pentene (urine), Unknown (1) (blood), Unknown (4) (blood), 3-Heptanone (urine), Unknown (13) (urine), 4-Heptanone (blood), 2-Heptanone (urine), Unknown (16) (urine), Dodecane (blood), Unknown (2) (blood), 2-Heptanone (blood), Unknown (5) (blood), 2-methyl 2-Propanol (blood), Unknown (8) (blood), 5-ethyl 2-methyl Octane (urine), Heptanal (blood), Unknown (3) (blood), Unknown (10) (blood), 1-Octene 3-ol (blood), 3-ethyl 3-Methylheptane (blood), Unknown (6) (blood), 3-Hexanone (urine), Tetradecane (blood), 2,4 dimethyl Decane (blood), Hexanal (blood), Pentanal (blood), 2,3 dihydro Furan (blood), Dodecane (urine), 2,3,5 trimethyl Hexane (urine), 2-pentyl Furan (blood), 3-Methyl Butanal (blood).

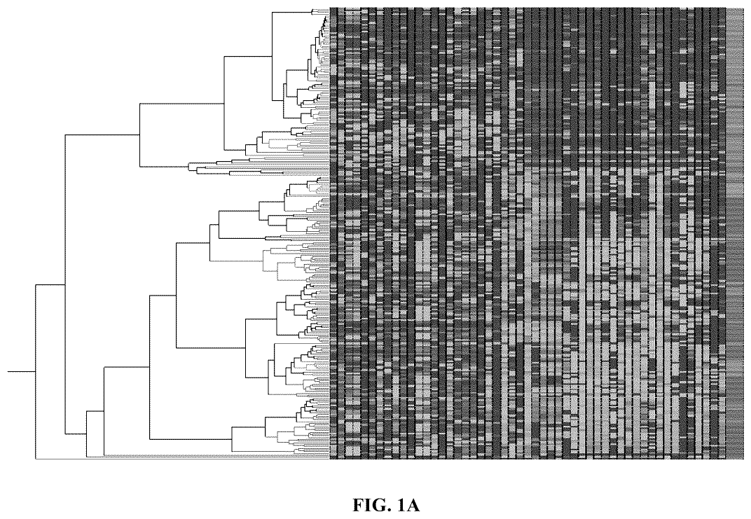

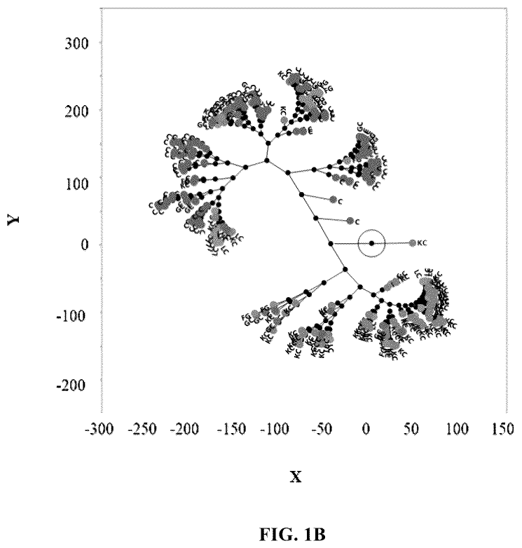

Owing to differences between the groups, multiple linear regression for each VOCs was used to explore any possible correlation between abundance and the covariates. Simple non-parametric Wilcoxon comparison showed that different volatiles showed significant differences between the test groups. Thus, a hierarchical clustering (using Ward's minimum variance method) was performed on raw VOC abundance data and resulted with clear clustering of the two main test groups, healthy subjects and kidney cancer patients. When analyzing blood samples model it was found that healthy and kidney cancer subjects were clearly clustered separately while other test groups with smaller size of subjects were not clustered, rather assimilated in the bigger groups. Urine clustering was found to be even less effective and most of the volatiles where not gathered to distinctive subgroups. Combining the two data sets of blood and urine improved the clustering (FIGS. 1A-1B) and besides the two main clusters of healthy and kidney cancer also the joint of sub groups of gastric cancer, lung cancer and fibro gastroscopy group can be seen (FIG. 1A).

TABLE 1
Tentative identification of volatiles detected in the headspace of
blood and urine samples using GC-MS

| VOC Number | Source | RT (min) | Tentative Identification |
|---|---|---|---|
| VOC 1 | Blood | 4.38035 | 2-methyl 2-propanol |
| VOC 2 | Blood | 5.2536 | Butanal |
| VOC 3 | Blood | 8.10495 | 3-methyl Butanal |
| VOC 4 | Blood | 10.94285 | pentanal |
| VOC 5 | Blood | 11.85008 | 2,4,4-trimethyl 1-Pentene |
| VOC 6 | Blood | 15.50652 | Butyl alcohol |
| VOC 7 | Blood | 15.90223 | Unknown (1) |
| VOC 8 | Blood | 16.33888 | Hexanal |
| VOC 9 | Blood | 17.21887 | 2,3,5-trimethyl Hexane |
| VOC 10 | Blood | 17.38257 | Unknown (2) |
| VOC 11 | Blood | 17.96927 | 2,3-dihydro Furan |
| VOC 12 | Blood | 19.11518 | 4-Heptanone |
| VOC 13 | Blood | 19.68147 | 2-Heptanone |
| VOC 14 | Blood | 19.9134 | Unknown (3) |
| VOC 15 | Blood | 20.04978 | Heptanal |
| VOC 16 | Blood | 21.30507 | Unknown (4) |
| VOC 17 | Blood | 21.69387 | Unknown (5) |
| VOC 18 | Blood | 22.42398 | 1-Octene-3-ol |
| VOC 19 | Blood | 22.77163 | 2-pentyl Furan |
| VOC 20 | Blood | 22.98993 | Unknown (6) |
| VOC 21 | Blood | 23.106 | Unknown (7) |
| VOC 22 | Blood | 23.5766 | Unknown (8) |
| VOC 23 | Blood | 24.43613 | 2,4-dimethyl Decane |
| VOC 24 | Blood | 24.57257 | 3-Ethyl-3-methylheptane |
| VOC 25 | Blood | 25.54128 | 2,4-dimethyl Decane |
| VOC 26 | Blood | 25.67778 | Unknown (9) |
| VOC 27 | Blood | 27.86063 | Dodecane |
| VOC 28 | Blood | 29.38212 | 2 Pyridinepropanoic acid, |
| | | | .alpha.-methyl- |
| | | | .beta.-oxo-, ethyl ester |
| VOC 29 | Blood | 29.40918 | Dodecane, 2,7,10-trimethyl- |
| VOC 30 | Blood | 29.68202 | Unknown (10) |
| VOC 31 | Blood | 29.94807 | Unknown (11) |
| VOC 32 | Blood | 31.96735 | Tetradecane |
| VOC 33 | Urine | 10.09008 | 2-pentanone |
| VOC 34 | Urine | 13.7877 | dimethyl disulfide |
| VOC 35 | Urine | 15.67722 | 3-hexanone |
| VOC 36 | Urine | 16.35942 | Unknown (12) |
| VOC 37 | Urine | 17.24612 | 2,3,5-trimethyl Hexane |
| VOC 38 | Urine | 17.74428 | 3-heptanone |
| VOC 39 | Urine | 17.9351 | 5-methyl 3-hexanone |
| VOC 40 | Urine | 18.9858 | 4-heptanone |
| VOC 41 | Urine | 19.42223 | Allyl Isothiocyanate |
| VOC 42 | Urine | 19.6678 | 2-Heptanone |
| VOC 43 | Urine | 21.34608 | Unknown (13) |
| VOC 44 | Urine | 22.47173 | dimethyl trisulfide |
| VOC 45 | Urine | 22.48517 | 2,3-Octanedione |
| VOC 46 | Urine | 23.5698 | 2,6-dimethyl Nonane |
| VOC 47 | Urine | 23.74728 | 2-ethyl hexanol |
| VOC 48 | Urine | 23.84268 | 1-(3-methylphenyl) Ethanone |
| VOC 49 | Urine | 24.4294 | 5-ethyl-2-methyl Octane |
| VOC 50 | Urine | 24.57942 | Dodecane |
| VOC 51 | Urine | 24.8659 | p-Cresol |
| VOC 52 | Urine | 25.2479 | 2-Nonanone |
| VOC 53 | Urine | 25.54828 | 4,5, dimethyl nonane |
| VOC 54 | Urine | 26.48948 | 3-Phenyl-2-pentene |
| VOC 55 | Urine | 26.99427 | pentyl benzene |
| VOC 56 | Urine | 27.64925 | menthol |
| VOC 57 | Urine | 27.85402 | Unknown (14) |
| VOC 58 | Urine | 27.92897 | 1-methyl-1-butenyl Benzene |
| VOC 59 | Urine | 29.17052 | Carvone |
| VOC 60 | Urine | 29.33412 | Unknown (15) |
| VOC 61 | Urine | 30.35745 | pentadecane |
| VOC 62 | Urine | 33.66593 | 2,3,5,8-tetramethyl-Decane |
| VOC 63 | Urine | 34.07535 | 2,4-bis(1,1-dimethylethyl) Phenol |
| VOC 64 | Urine | 35.24177 | Unknown (16) |

In order to improve these results, average abundance values of each VOC in each group were examined rather than the raw data, which significantly improved the clustering. VOCs from blood were able to create individual clusters for each group where healthy and gastric cancer were sub grouped, same as lung and kidney cancer, while fibro gastroscopy group was separated from all. VOCs from urine sample clustered healthy and lung cancer as subgroups, gastric and kidney cancer as subgroups and fibro gastroscopy as separate group. Interestingly, the combination of both data sets of average VOCs abundance from blood and urine samples clustered the groups perfectly where both healthy controls and fibro gastroscopy groups were clustered together as sub groups and different cancer types were clustered separately. It could be therefore concluded that VOCs can be used to distinguish between cancer and non-cancer subjects and between different cancer types with high accuracy, when using a combination of blood and urine samples.

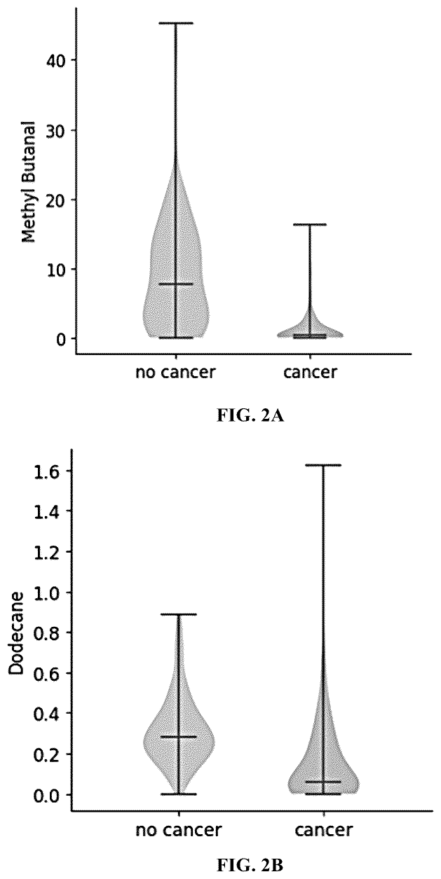

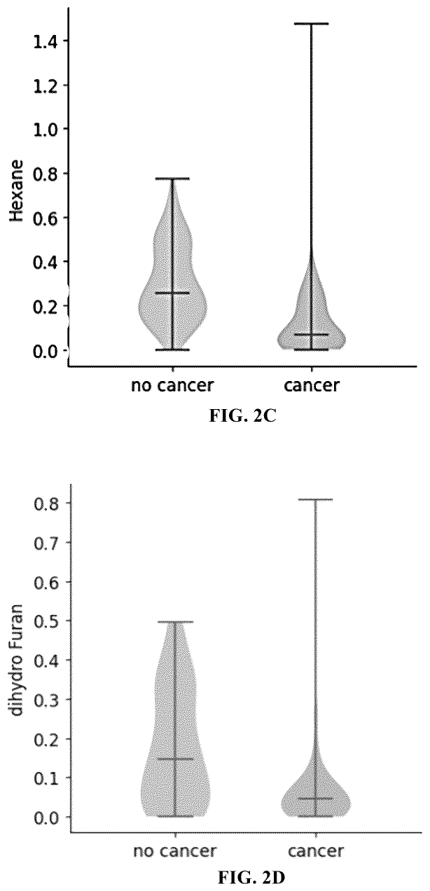

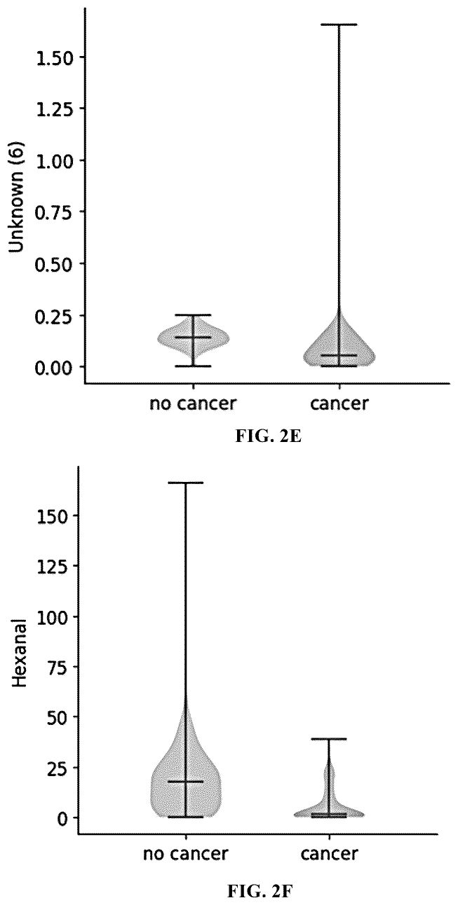

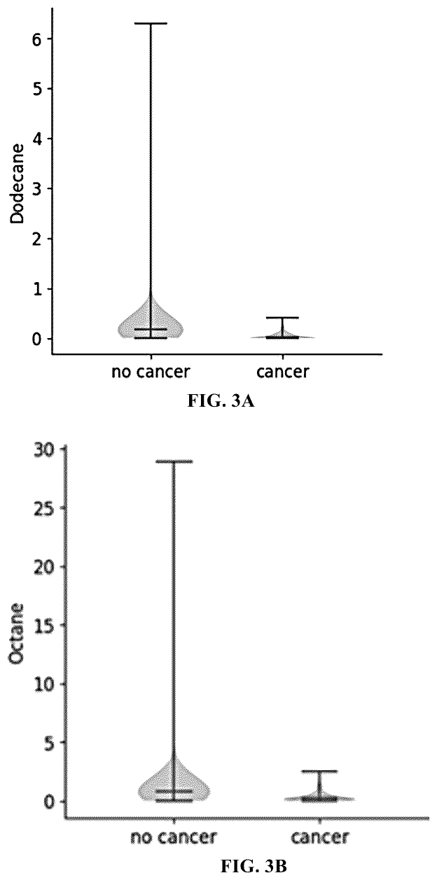

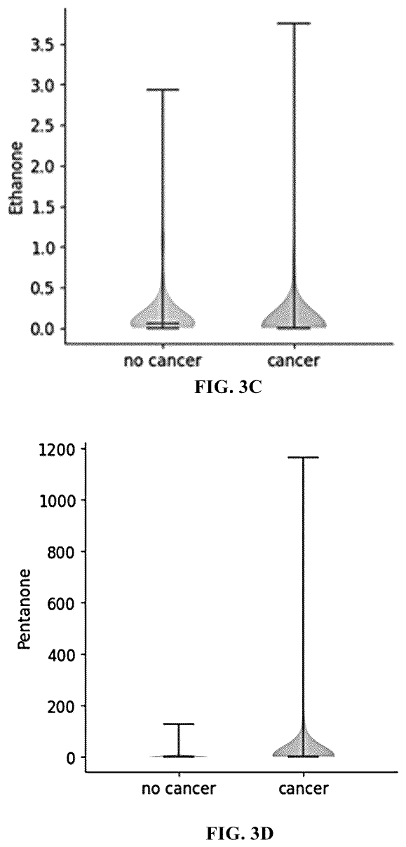

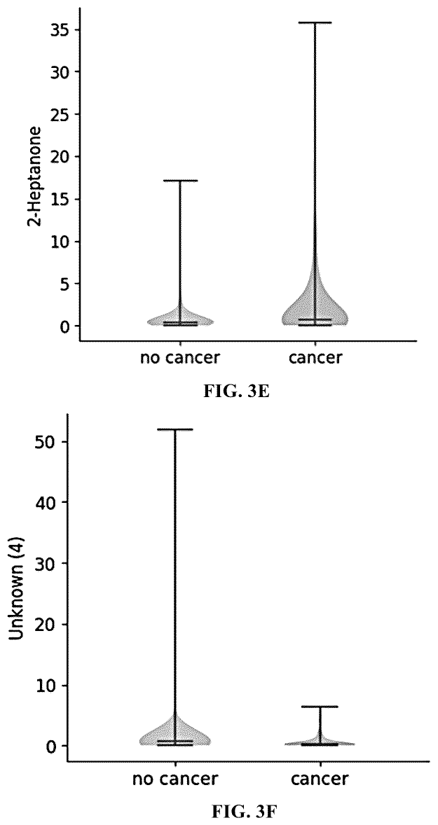

Further, an artificially intelligent model based on machine learning has been developed that can accurately detect cancer and monitor its progress. First, a two-class classification task has been considered, cancer versus non-cancer., thus grouping all types of cancers into a single “cancer” class. A statistical analysis (Wilcoxon rank-sum test) has been run to determine which features could be discriminative between the two classes: cancer and healthy patients. For the blood data, 26 features got p value lower than 0.05, while for the urine data 16 features had p value lower than the threshold. FIGS. 2A-2F show the distribution of six discriminative features for the blood data, while FIGS. 3A-2F show the distribution of six discriminative features for the urine data.

Three models have been developed, one using the blood data, a second one using the urine data. Finally, the last model combined the features from blood and urine data. In order to fairly compare all the models, only the patients with the combined data were used for the classification task (n=255). For each model, a Random Forest (RF) was trained, as detailed hereinabove. For the blood model 26.8±1.0 (mean±std) features were selected, and for the urine model 17.2±1.7 were selected. Finally, for the combined model, 41.4±2.1 features were selected, including 26.2±1.0 features from blood data, and 15.2±1.1 features from urine data. Table 2 presents the results for the three models proposed for discriminating cancer from non-cancer subjects. Blood VOCs pattern-based model showed higher performances than urine VOCs pattern-based model (i.e., discrimination accuracy of 92% and 82%, respectively). While sensitivity and specificity of blood based models were relatively high (90% and 89%), urine based model showed low sensitivity and specificity (78% and 70%) for discrimination. The combined model improved all parameters and showed the highest values of discrimination accuracy, sensitivity and specificity (94%, 92% and 91% respectively). Model accuracy was high in all models as seen from the AU ROC and F₁ parameters.

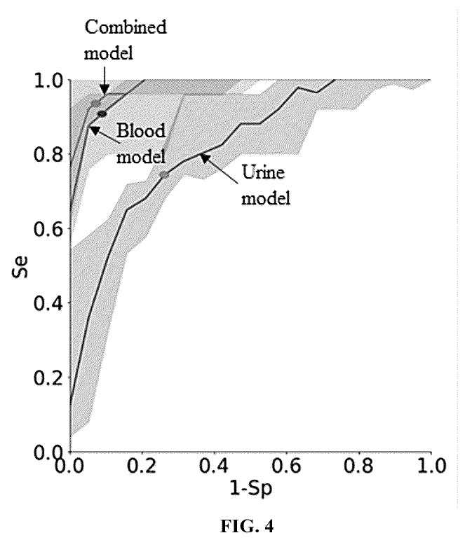

This is also emphasized in FIG. 4, which shows ROC curves. The points on each ROC curve were chosen to maximize the F1 score. For the blood model, a total of 23 patients were mis-classified (9% of all patients). 7 with no cancer (5% of all non-cancer), 9 with gastric cancer (45% of all gastric cancer), 5 with kidney cancer (6% of all kidney cancer) and 2 with lung cancer (10% of all lung cancer). For the urine model (which performed less well), a total of 51 patients were mis-classified (20% of all patients). 24 with no cancer (18% of all non-cancer), 6 with gastric cancer (30% of all gastric cancer), 16 with kidney cancer (19% of all kidney cancer) and 5 with lung cancer (25% of all lung cancer). For the combined model, a total of 21 patients were mis-classified (8% of all patients). Among them, 5 have no cancer (3% of all non-cancer), 8 have gastric cancer (40% of all gastric cancer), 6 have kidney cancer (7% of all kidney cancer), and 2 has lung cancer (10% of all lung cancer). Combining the two types of data (blood and urine) improved the classification from 0.91±0.08 for blood and 0.83±0.06 for urine to 0.93±0:03. When removing the demographic features: age, sex and smoking status, there was a drop of 0.02 for the ROC, 0.01 for the F1 score.

TABLE 2
Results of the different models proposed for discriminating
cancer from non-cancer volunteers

| | AU ROC | F₁ | Se | Sp | Acc |
|---|---|---|---|---|---|
| Blood | 0.91 ± 0.08 | 0.92 ± 0.05 | 0.90 ± 0.04 | 0.89 ± 0.06 | 0.92 ± 0.03 |
| Urine | 0.83 ± 0.06 | 0.82 ± 0.03 | 0.78 ± 0.04 | 0.70 ± 0.00 | 0.82 ± 0.03 |
| Combined | 0.93 ± 0.03 | 0.92 ± 0.05 | 0.92 ± 0.03 | 0.91 ± 0.02 | 0.94 ± 0.05 |

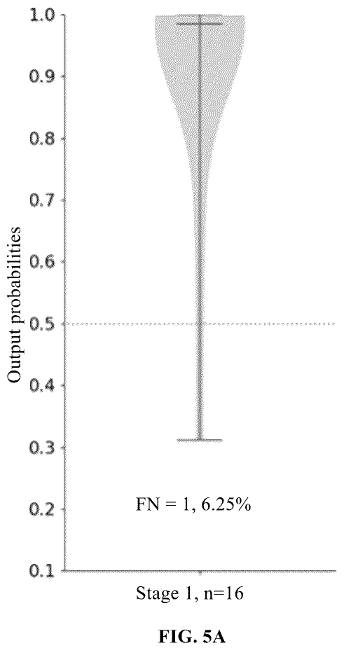

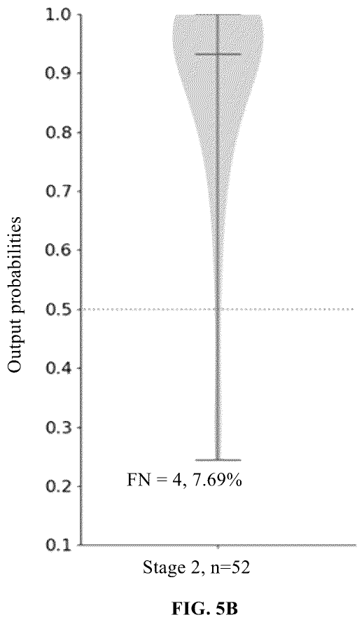

FIGS. 5A-5C show the distribution of the output probabilities for three different stages (degree of severity) of cancer. The stage of cancer was available for only 87 patients out of 255, and 52 patients among them have cancer of stage 2 so there may be a bias in that respect. Nevertheless, it can be noticed that no patient with cancer of stage 3 was missed.

TABLE 3
Results of the different models proposed for discriminating gastric cancer
patients from fibro gastroscopy volunteers

| | Auroc | F₁ | Se | Sp | Acc |
|---|---|---|---|---|---|
| Blood | 0.66 ± 0.13 | 0.72 ± 0.14 | 0.33 ± 0.26 | 1.00 ± 0.00 | 0.92 ± 0.02 |
| Urine | 0.66 ± 0.10 | 0.73 ± 0.11 | 0.33 ± 0.22 | 0.95 ± 0.02 | 0.88 ± 0.01 |
| Combined | 0.83 ± 0.12 | 0.88 ± 0.10 | 0.00 ± 0.26 | 1.00 ± 0.00g | 0.06 ± 0.03 |

It has further been shown (table 3) that it was possible to discriminate between cancer and non-cancer subjects showing gastric cancer symptoms. Blood VOCs pattern-based model showed higher performances than urine VOCs pattern-based model, although both presented high discrimination accuracy of 92% and 88% respectively. Models' sensitivity was very low, unlike high specificity of 100% in blood based model and 95% in urine based model. The combined model improved all parameters and showed the highest values of discrimination accuracy, sensitivity and specificity (96%, 66% and 100% respectively).

### Example Diagnosing Cancer by Analyzing VOCs in Blood and Urine Samples by Chemically Sensitive Sensors

Collection of Blood and Urine Headspace: Samples inserted into headspace glass vials closed for 1 hour before sampling and then heated on a hot plate to 70° C. for 15 min before sampling. Samples were than subjected to pre-concentration on Tenax TA tube. A Tenax TA tube was inserted into the tube holder and N2 was streamed through stainless steel needles (5 cm, 14 g) in 50 ml/min for 30 minutes through the system by way of a hydrocarbon and humidity filter (super Clean™ gas filters (SGT)). Tubes were transferred to TD-GC-E-Nose system.

Preparation of gold nanoparticle sensors: Gold nanoparticles (NPs) coated with organic layers can be synthesized using the two-phase Brust method (Brust et al. 2002, Colloids and Surfaces A, 202, 175-186; Brust et al. 1994, Journal of the Chemical Society, Chemical Communications, 801-802). AuCl₄ was transferred from aqueous HAuCl₄·XH₂O solution (25 mL, 36.5 mM) to a toluene solution by phase-transfer reagent tetraoctylammonium bromide (TOAB; 80 mL, 34.3 mM). After stirring, the organic phase was isolated and an excess of the chosen thiol was added to the solution. In order to receive a monodispersed solution of Au NPs, the molar ratio of HAuCl₄·XH₂O to thiol is varied between 1:1 and 1:10 depending on the type of thiol. After stirring for 10 min, an aqueous solution of reducing agent sodium borohydride (NaBH4), in large excess (25 mL, 0.4 M) is added to the solution. The reaction occurred by stirring at room temperature for 3 hours, producing a dark-brown solution. After separating the solution from the aqueous phase, the resulting solution was subjected to solvent removal in a rotary evaporator at 40° C. and followed by addition ethanol to the dried solution. The samples were kept in freezer for several days until sedimentation of the particles and afterwards were transferred to a centrifuge at 400 rpm and a temperature of 4° C. for additional sedimentation of the particles. The resulting solution is subjected to solvent removal in a rotary evaporator. The NPs were purified from free thiol ligands by repeated extractions. The coated gold nanoparticles were prepared at a range of concentration between 1 mg/mL and 500 mg/mL.

The coated gold nanoparticles were then dispersed in either toluene or ethanol. Chemiresistive layers were formed by drop-casting the solution onto microelectronic transducers, until a resistance of several MS/was reached.

Preparation of sensors of functionalized single walled carbon nanotubes: Sensors of functionalized single walled carbon nanotubes were formed by drop-casting a solution of SWCNTs (from ARRY International LTD, Germany; ˜30% metallic, ˜70% conducting, average diameter=1.5 nm, length=7 mm) in dimethylformamide (DMF, from Sigma Aldrich Ltd., >98% purity) onto the pre-prepared electrical transducers. The sensors were based on an electrically continuous random network of SWCNTs (U.S. Pat. Nos. 8,366,630; 8,481,324; the contents of each of which are hereby incorporated in their entirety). After the deposition, the device was slowly dried overnight under ambient conditions to enhance the self-assembly of the SWCNTs and to afford the evaporation of the solvent. The procedure was repeated until a resistance of 100 KΩ to 10 MΩ was obtained. The SWCNT sensor was organically functionalized with a polycyclic Aromatic Hydrocarbon (PAH) derivative hexa-perihexabenzocoronene.

Intelligent nanosensor array (TD-GC-E-Nose system): A stainless-steel cell for exposure contained an array of 40 nanomaterial-based sensors mounted on a customized polytetrafluoroethylene circuit. The sensors included:
gold-nanoparticles (organically-stabilized spherical Au nanoparticles, core diameter 3-4 nm) capped with different organic layers, including: tert-dodecanethiol; butanethiol; 4-chlorobenzenemethanethiol; 4-tert-butylbenzenethiol; dibutyl disulfide; 2-naphthalenethiol; 2-nitro-4-(trifluoromethyl)benzenethiol; dodecanethiol; octadecanethiol; decanethiol; 2-ethylhexanethiol; 3-ethoxythiophenol; benzylmercaptan; and hexanethiol;
conducting polymers, including: diketopyrrolopyrrole-anthracene (FAF); diketopyrrolopyrrole-naphthalene (TNT); diketopyrrolopyrrole-benzothiadiazole (TBT) and hexyldecyl-substituted poly(diketopyrrolopyrrole) (PDPPHD);
conductive polymer composites, including: CB/poly(propylene-urethaneureaphenyl-disulfide) PPUU-2S; CB/poly(propylene-urethaneureaphenyl-disulfide) PPUU-2S mixed with poly(urethane-carboxyphenyl-disulfide) PUC-2S (CB/(PUC-2S/PPUU-2S) composite); and CB/poly(2-hydroxypropyl methacrylate) mixed with polyethyleneimine (CB/(PPMA/PEI) Composite); and
single-walled carbon nanotubes (RN-SWCNTs) coated with: Polycyclic Aromatic Hydrocarbon 3 (PAH-3) and crystal hexa-perihexabenzocoronene ((HBC) C12).

Analyzing the samples: To transfer the VOCs trapped on the absorption materials, the samples were thermally desorbed at 270° C. in an auto-sampler desorption system (TD20; Shimadzu Corporation, Japan). The desorbed samples were temporarily stored in a stainless-steel sampling loop at 150° C. In parallel, the chamber containing the sensors was kept under vacuum conditions (˜30 mTor) until the sample had been transferred into the chamber. The remaining volume was filled with pure N2 until it reached atmospheric pressure. A Keithley data logger device (model 2701 DMM) was used to acquire resistance readings from the sensor array sequentially. The whole system was controlled by a custom-made LabView program. The following sequence was maintained for each sample measurement: 5 min in vacuum, 5 min pure N2 gas, 5 min vacuum, 5 min sample or calibration exposure, followed by 5 min vacuum and 3 min pure N2 gas, and finally 3 min in vacuum. To supervise sensor functionality during the experiment, and to overcome possible sensor response drift, a fixed calibration gas mixture containing 11.5 ppm isopropyl alcohol, 2.8 ppm trimethylbenzene and 0.6 ppm 2-ethyl-hexanol was exposed to the sensors daily. The calibration was generated using a commercial permeation/diffusion tube dilution (PDTD) system (Umwelttechnik MCZ, Germany). The system controls the concentration of the VOCs. Purified dry nitrogen (99.999%) from a commercial nitrogen generator (N-30, On Site Gas Systems, USA) equipped with a nitrogen purifier was used as a carrier gas. The calibration mixture was absorbed on a clean Tenax TA tube for 5 min. Several features can be extracted from the sensor's signal upon exposure, including Area under curve, delta R peak, delta R middle and delta R end. The last 3 features are based on the difference between the baseline resistance, usually during vacuum, and the resistance during the response towards the exposure: peak point, middle part and the end part of the signal.

### Example 6—Diagnosing Cancer by Analyzing VOCs in Blood and Urine Samples by Chemically Sensitive Sensors—Data Analysis

Discriminant Function Analysis (DFA): Data obtained from the sensors were analyzed using DFA, which is a statistical method used when the groups to be discriminated are defined (labeled) before being analyzed. The input variables for DFA are the features extracted from sensors' responses towards the headspace samples. The method determines either a linear or quadratic combination of the input variables in order to receive minimum variance within each group and maximum variance between the groups. The decision on either linear or quadratic model was based on homogeneity of the variance-covariance matrices of the tested groups according to statistical tests, e.g. Bartlett's test. Leave-one-out cross-validation was used to calculate the success of the classification in terms of the numbers of true positive (TP), true negative (TN), false positive (FP), and false negative (FN) predictions. Given k measurements, the model was computed using k−1 training vectors. All possibilities of leave-one-sample-out were considered, and the classification accuracy was estimated as the average performance over the k tests. For pattern recognition and data classification, Python (Python Software Foundation) was used. In addition, a ratio of 1:6 between samples and explanatory variables was kept to reduce the chance of over fitting. Correct classification of the data points was counted and presented as sensitivity and specificity values according to Equations (1)-(5):

Sensitivity=TP/(TP+FN) Equation (1)

Specificity=TN/(FP+TN) Equation (2)

Accuracy=(TP+TN)/(TP+TN+FP+FN) Equation (3)

Positive predictive value(PPV)=TP/(TP+FP) Equation (4)

Negative predictive value(NPV)=TN/(TN+FN) Equation (5)
where TP=true positive, FN=false negative, TN=true negative and FP=false positive.

### Example 7—Diagnosing Cancer by Analyzing VOCs in Blood and Urine Samples by Chemically Sensitive Sensors—Results

The present experiment was designed to show that an array of chemically sensitive sensors can be used to detect cancer patients and distinguish them from non-cancer ones when analyzing blood and urine samples. The test samples were obtained from the patients as described in Example 1.

Cancer is a complex condition involving most of the systems in the body, making it very difficult to be associated with just one unique biomarker, which is the main disadvantages of known liquid biopsy protocols. Therefore, using a cross-reactive approach in which a combination of nonselective sensors is used can overcome the lack of specific markers. In this approach, each sensor responds differently to individual VOCs or to a pattern of VOCs in the sample, allowing the evaluation of the VOC pattern in a qualitative manner, while selectivity is achieved by predictive methods that are based on machine learning models. The samples headspace was exposed to an array of chemiresistors based on organically stabilized spherical gold nanoparticles (GNPs) with a core diameter of 3-4 nm, 2D random networks of single-walled carbon nanotubes (RN-SWCNTs) capped with different organic layers, and polymeric composites. Then, using machine-learning methods an AI model was trained and tested to discriminate between the different groups.

Discriminate Factor Analysis (DFA) from 4-15 sensor features was used to separate cancer patients from controls. The analysis was based on blood and urine sampling and included a total of 600 samples (300 urine and 300 blood), of which 150 were from confirmed cancer patients and 150 from non-cancer and healthy volunteers. The evaluation of the performance of the quadratic DFA model was based on a randomly selected blinded-test group (30% of the total dataset). First, grouping of all cancer groups to one “cancer” group and fibrogastroscopy and healthy volunteers to “non-cancer” group was performed. Three optional models were created based on blood data, urine data, and combined data. The differences of the sensors' response to cancer or non-cancer groups can be seen in the DFA models (Tables 4-6). In the training phase, the results yielded high scores for all three models: including 93.3% accuracy, 88.5% sensitivity, and 98% specificity in blood, and 86.1% accuracy, 95.8% sensitivity and 76.7% specificity, in urine (Table 4). The combined model did not improve the performances and received 88.7% accuracy, 95.8% sensitivity and 81.6% specificity (Table 4). The analysis of the blinded-test group (30%) resulted in better performances and the combined model increased accuracy, sensitivity and specificity up to 97.6%, 97.7% and 97.4% respectively (Table 5).

Three potential confounding factors and their influence on the results of the models were evaluated. These included age, sex and smoking status. Their influence was based on the accuracy of the models used to discriminate between cancer and non-cancer groups. Table 6 gives the accuracies, which were 30-56.6%, i.e., mostly arbitrary. Thus, no significant difference within each of the confounding factors was found. Age effect may be as a result on un-even cohort.

TABLE 4
Training phase results of the different sensor based DFA models
proposed for discriminating cancer from non-cancer volunteers.

| No Cancer vs. | Training | | |
|---|---|---|---|
| Cancer | Accuracy (%) | Sensitivity (%) | Specificity (%) |
| Blood | 93.3 | 88.5 | 98 |
| Urine | 86.1 | 95.8 | 76.7 |
| Combined | 88.7 | 95.8 | 81.6 |

TABLE 5
Blinded-test phase results of the different sensor based DFA models
proposed for discriminating cancer from non-cancer volunteers.

| No Cancer vs. | Test | | |
|---|---|---|---|
| Cancer | Accuracy (%) | Sensitivity (%) | Specificity (%) |
| Blood | 94.4 | 88.8 | 100 |
| Urine | 90.4 | 95.5 | 84.6 |
| Combined | 97.6 | 97.7 | 97.4 |

TABLE 6
Effect of the confounding factors on the different
sensor based DFA models proposed for
discriminating cancer from non-cancer volunteers.

| No Cancer vs. | Confounding Factors Accuracy (%) | | |
|---|---|---|---|
| Cancer | Age | Sex | Smoke |
| Blood | 30 | 49.4 | 50 |
| Urine | 38.3 | 56.6 | 48 |
| Combined | 33.2 | 46.5 | 54.5 |

Further analysis targeting sub-populations was carried out, which included discrimination between different kinds of cancer including gastric cancer, kidney cancer and lung cancer. In all three models, the accuracies of the training sets of the different models ranged between 75-91.5% while test sets ranged between 80-100% (Tables 7-9). The combined model consistently showed better performance, for example, test accuracy for discriminating kidney cancer from gastric cancer was 92% in blood 91.6% in urine and reached 97.2% in the combined model (Table 7). For all the models the discrimination accuracy for the confounding factors was approximately 50%. It can therefore be concluded that the confounding factors do not affect the model performances.

TABLE 7
Results of the different sensor based DFA
models proposed for discriminating
Gastric cancer and Kidney cancer patients.

| Gastric | | | | | |
|---|---|---|---|---|---|
| cancer vs. | Training | Test | Confounding Factors | | |
| Kidney | Accuracy | Accuracy | Accuracy (%) | | |
| cancer | (%) | (%) | Age | Sex | Smoke |
| Blood | 85 | 92 | 48.8 | 58.4 | 46.4 |
| Urine | 88.8 | 91.6 | 55.5 | 63.2 | 37.6 |
| Combined | 90.2 | 97.2 | 52.9 | 62.3 | 48.7 |

TABLE 8
Results of the different sensor based DFA models proposed
for discriminating Gastric cancer and Lung cancer patients.

| Gastric | Training | Test | Confounding Factors | | |
|---|---|---|---|---|---|
| cancer vs. | Accuracy | Accuracy | Accuracy (%) | | |
| Lung cancer | (%) | (%) | Age | Sex | Smoke |
| Blood | 91.4 | 93.7 | 52.9 | 62.7 | 62.7 |
| Urine | 75 | 80 | 61.7 | 63.8 | 48.9 |
| Combined | 90.6 | 100 | 57.4 | 63.8 | 65.9 |

TABLE 9
Results of the different sensor based DFA models proposed for
discriminating Kidney cancer and Lung cancer patients.

| Kidney | Training | Test | Confounding Factors | | |
|---|---|---|---|---|---|
| cancer vs. | Accuracy | Accuracy | Accuracy (%) | | |
| Lung cancer | (%) | (%) | Age | Sex | Smoke |
| Blood | 94.1 | 97.3 | 50.8 | 62 | 46.7 |
| Urine | 91.4 | 100 | 45.7 | 40.7 | 66.9 |
| Combined | 91.5 | 100 | 49.1 | 42.3 | 66.9 |

Because of the relatively small number of samples with cancer stage data, a simple t-test was preformed to test the potential of the nanoarray to also stage and monitor cancer progression. It has been surprisingly found that certain sensors were able to distinguish alone between different cancer stages with p value lower than 0.05. For example, the sensor comprising GNPs coated with octadecanethiol discriminated between blood samples of kidney cancer early and advanced stage with p value of the same sensor discriminated between blood samples of lung cancer early and advanced stage with p value of 0.0002. In urine samples said sensor discriminated between lung cancer early and advanced stage with p value of 0.0406.

### Example 8—Diagnosing Cancer by Analyzing VOCs in Blood and Urine Samples by a Portable Device Comprising Chemically Sensitive Sensors

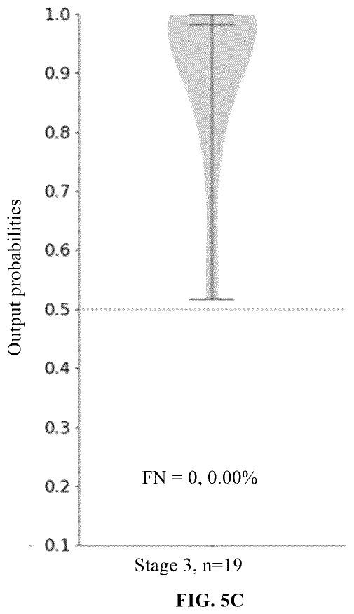

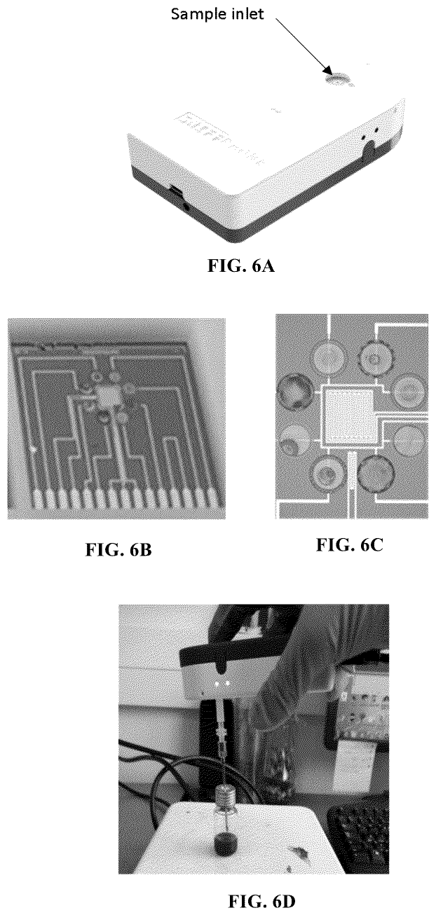

Nanosensor-based portable device: A portable hand-held device (shown in FIG. 6A), comprising an array of chemically sensitive sensors was further used to detect cancer patients and distinguish them from non-cancer ones when analyzing blood and urine samples. Without wishing to being bound by theory or mechanism of action, it is contemplated that the operating principle of this device lies in the change of resistance of the sensors upon exposure to a particular mixture of VOCs, therefore allowing the device to be trained to recognize a particular disease with no need of prior processing of the samples.

The nanomaterial-based sensor array that was used in the portable device contained cross-reactive, chemically diverse chemiresistors that were based on spherical gold nanoparticles (GNPs, core diameter: 3-4 nm) coated with the following organic ligands: dodecanethiol, 4-tert methyl-benzenethiol, 2-ethylhexanethiol, decanethiol, tert-dodecanethiol, 4-chloroben-zenemethanethiol, 3-ethoxytiophenol, and hexanethiol). Organically capped GNPs were synthesized as described in Example 5.

Circular inter-digitated platinum electrodes were deposited by an electron-beam evaporator (Evatec BAK501) on a silicon wafer capped with thermal silicon oxide film of 1 micron (purchased from Nova electronic materials, LLC, USA). The outer diameter of the circular electrode area was 1000 μm; the gaps between two adjacent electrodes and the width of each electrode were both 10 μm. The chip was designed to include eight sensors. Each of the eight electrodes had a ring (micro-barrier) around it. This ring was 2.5 μm in height and 100 μm in width and was designed to hold the drop casted GNP emulsion by providing a physical barrier to its dispersion. The micro-barrier was fabricated in the clean room facilities by a photolithography process. The chip and the sensor array are shown in FIGS. 6B and 6C.

Measurement of the headspace was done by connecting the device opening to a stainless steel needle (5 cm, 14 g), that was inserted into the headspace vial up to 0.5 cm from the liquid (FIG. 6D). The measurement protocol consisted of three steps: baseline (during the first 5s the pump would automatically trap room air and measure it for 12.75s), sample reading (after connecting the device to the headspace vial it would pump for 1s headspace from the vial and measure it for 12.75s) and cleaning (after disconnecting the device, the baseline step was repeated). To ensure validity of the results and to reduce artifacts from the environment, at the beginning of each sampling day a test was performed on an empty vial followed by a calibration vial (100 ppb EPA 524 Internal Standard Mix (sigma) in HPLC graded Methanol (>99.9%)) with the same sampling protocol. In addition to the features mentioned in Example 5, including Area under curve, delta R peak, delta R middle and delta R end. here also resistance curve slope was extracted. The portable device was connected to a notebook computer.

### Example 9—Diagnosing Cancer by Analyzing VOCs in Blood and Urine Samples by a Portable Device Comprising Chemically Sensitive Sensors Results

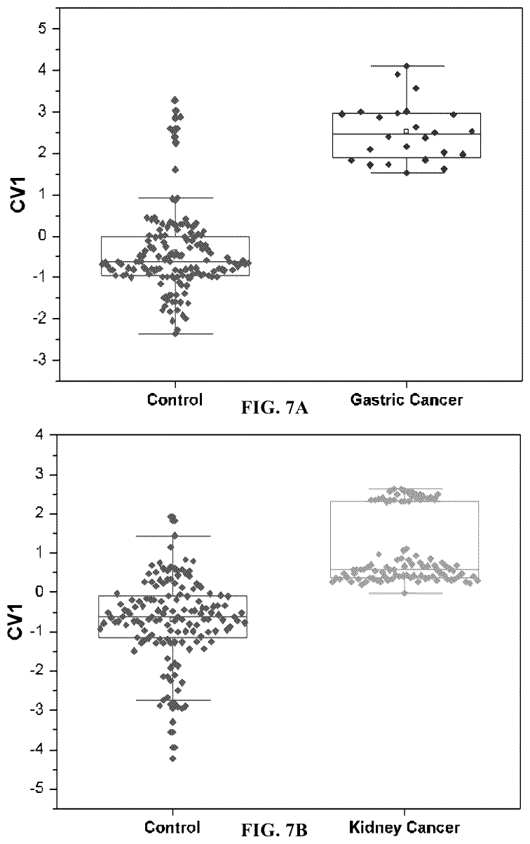

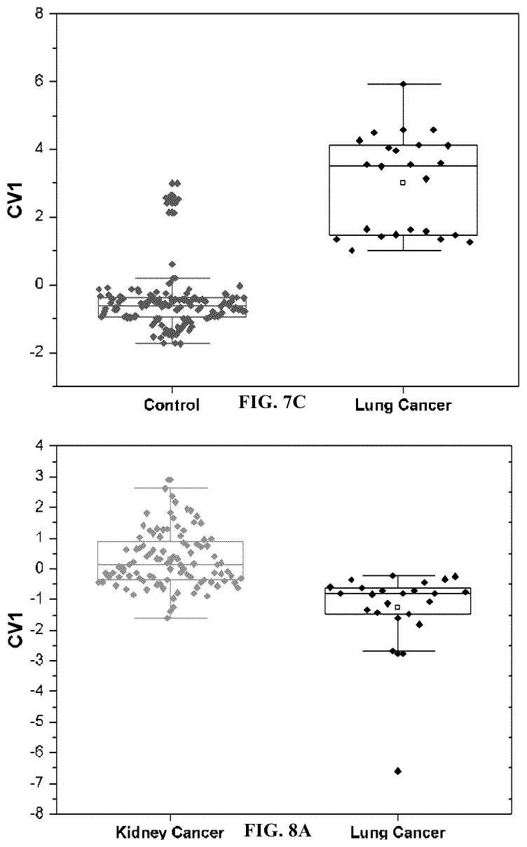

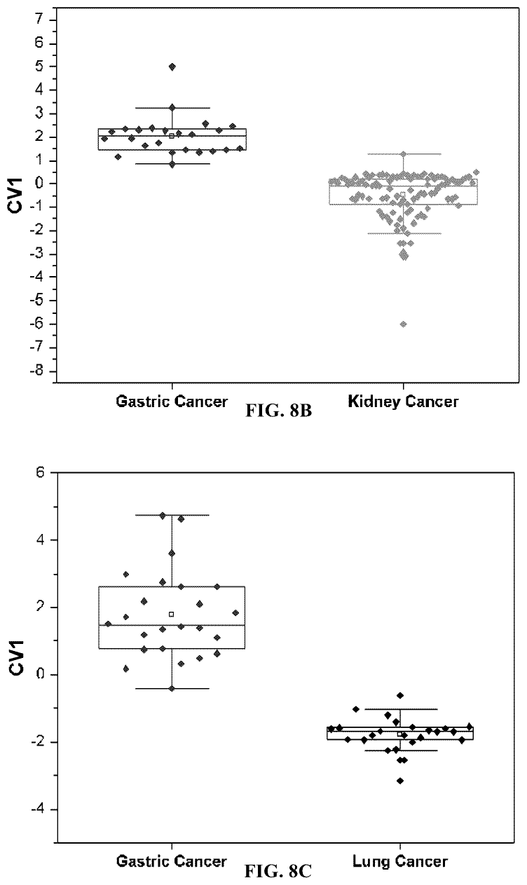

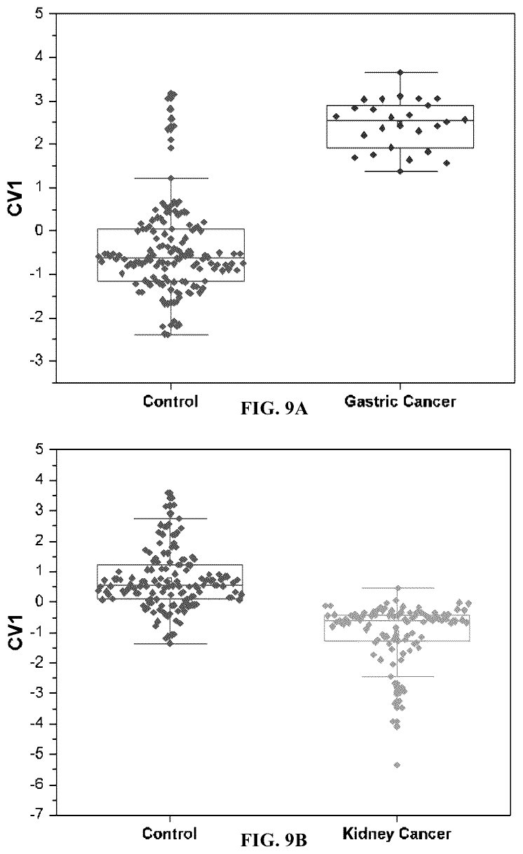

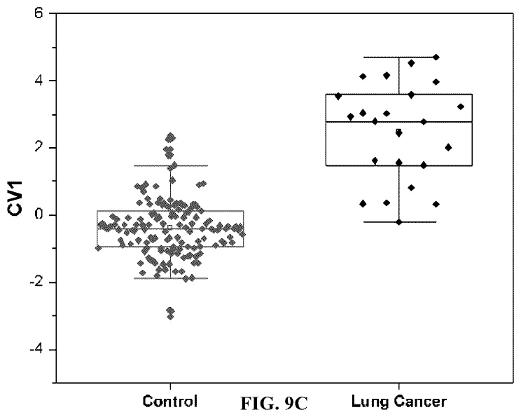

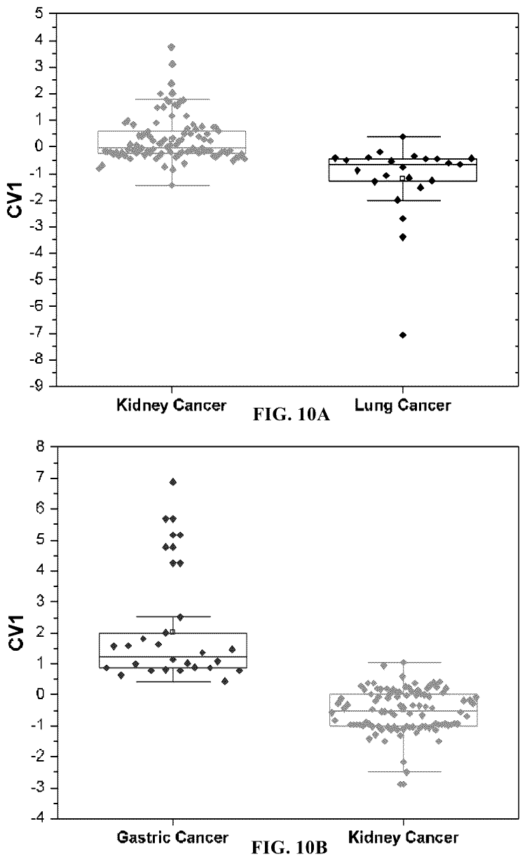

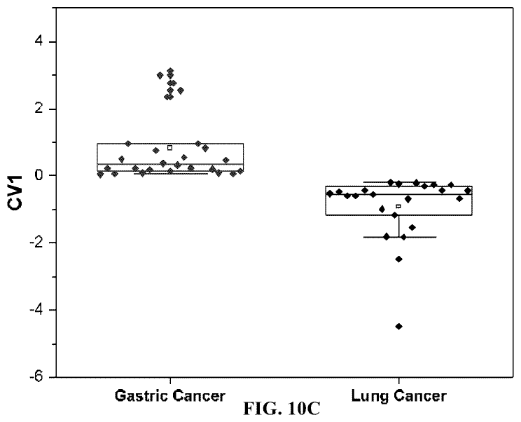

The portable device was placed in direct contact with the headspace of the blood and urine headspace, thereby obviating the need to use any absorbent material or pre-concentration technique. The device, which included 8 nanomaterial-based sensors, was placed above the sample in a sterile environment and an aliquot of the sample headspace comprising very low concentrations of the cancer biomarker VOCs was drawn into the device for real-time response. Post processing analysis by linear DFA resulted in a leave-one-out validation model with accuracies ranging from 88.2-94% in blood (FIGS. 6A-6C) and 88.3-94% in urine samples (FIG. 8A-8C) discriminating gastric, kidney and lung cancer from non-cancer volunteers. The sensitivity of the models ranged between 83.3-100% in blood (FIGS. 7A-7C) and 78.3-100% in urine samples (FIGS. 9A-9C); and the specificity of the models ranged between 81-93.2% in blood (FIGS. 7A-7C) and 85.6-93% in mine samples (FIGS. 9A-9C) when discriminating between cancer patients and non-cancer subjects. Models' accuracies ranged from 82.5-99% in blood (FIGS. 8A-8C) and 87.2-100% in urine samples (FIGS. 10A-10C) discriminating between different cancer types. The sensitivity of the models ranged between 79-100% in blood (FIGS. 8A-8C) and 92.15-100% in urine samples (FIGS. 10A-10C), while the specificity of the models ranged between 83-100% in blood (FIGS. 8A-8C) and 65.2-100% in urine samples (FIGS. 10A-10C) when discriminating between different types of cancer.

Additional data analysis was performed with the data obtained from the portable device (tables 10-15). The new analysis (performed with Python) does not include all the possible features obtained from the portable device measurement. It can be seen that the combined model improved discrimination accuracy as compared to the blood model and urine model, between cancer patients and healthy subjects (table 11), as well as between certain types of cancer, e.g., between gastric cancer and urine cancer, where the discrimination accuracy was 100% with the combined model (table 14).

TABLE 10
Training phase results of the different portable sensor based DFA models
proposed for discriminating cancer from non-cancer volunteers.

| | Training | | |
|---|---|---|---|
| No Cancer vs. | Accuracy | Sensitivity | Specificity |
| Cancer | (%) | (%) | (%) |
| Blood | 83.3 | 82 | 96 |
| Urine | 89.85 | 83.5 | 96.15 |
| Combined | 86.3 | 78.43 | 94.11 |

TABLE 11
Blinded-test phase results of the different portable sensor based DFA
models proposed for discriminating cancer from non-cancer volunteers.

| No Cancer vs. | Test | | |
|---|---|---|---|
| Cancer | Accuracy (%) | Sensitivity (%) | Specificity (%) |
| Blood | 89.77 | 81.4 | 97.77 |
| Urine | 92.22 | 91.1 | 93.3 |
| Combined | 94.3 | 93.02 | 95.5 |

TABLE 12
Effect of the confounding factors on the different
portable sensor based DFA models proposed
for discriminating cancer from non-cancer volunteers.

| No Cancer vs. | Confounding Factors Accuracy (%) | | |
|---|---|---|---|
| Cancer | Age | Sex | Smoke 5 |
| Blood | 65.87 | 49.48 | 42.66 |
| Urine | 33.3 | 51.17 | 57.57 |
| Combined | 34.13 | 48.62 | 55.17 |

TABLE 13
Results of the different portable sensor based DFA models proposed
for discriminating Gastric cancer and Kidney cancer patients

| Gastric | | | | | |
|---|---|---|---|---|---|
| cancer vs. | Training | Test | Confounding Factors | | |
| Kidney | Accuracy | Accuracy | Accuracy (%) | | |
| cancer | (%) | (%) | Age | Sex | Smoke |
| Blood | 95.22 | 97.22 | 52.5 | 63.3 | 44.16 |
| Urine | 93.1 | 92.1 | 48 | 60 | 44.8 |
| Combined | 98.82 | 94.59 | 55 | 60.8 | 45 |

TABLE 14
Results of the different portable sensor based DFA models proposed
for discriminating Gastric cancer and Lung cancer patients.

| Gastric | Training | Test | Confounding Factors | | |
|---|---|---|---|---|---|
| cancer vs. | Accuracy | Accuracy | Accuracy (%) | | |
| Lung cancer | (%) | (%) | Age | Sex | Smoke |
| Blood | 96.96 | 93.33 | 58.33 | 66.66 | 64.58 |
| Urine | 94.11 | 93.33 | 53.06 | 59.18 | 65.3 |
| Combined | 100 | 100 | 57.4 | 65.95 | 70.2 |

TABLE 15
Results of the different portable sensor based DFA models proposed
for discriminating Kidney cancer and Lung cancer patients

| Kidney | Training | | Confounding Factors | | |
|---|---|---|---|---|---|
| cancer vs. | Accuracy | Test Accuracy | Accuracy (%) | | |
| Lung cancer | (%) | (%) | Age | Sex | Smoke |
| Blood | 86.9 | 86.1 | 59.16 | 48.33 | 60 |
| Urine | 92.94 | 97.3 | 54.09 | 44.26 | 60.65 |
| Combined | 96.42 | 97.3 | 53.78 | 42.85 | 60.5 |

### Example 10—Diagnosing Cancer by GC-MS and Chemically Sensitive Sensors—Additional Study

TABLE 16
VOCs significantly discriminating between healthy and cancerous patients and between different
types of cancer. VOCs obtained from blood and urine headspace of volunteers.

| | | | | | | | |
|---|---|---|---|---|---|---|---|
| Tentative | C vs | C vs | C vs | FG vs | GC vs | GC vs | LC vs |
| identification | KC | LC | GC | GC | LC | KC | KC |

Blood samples

| | | | | | |
|---|---|---|---|---|---|
| Hexane | | p < 0.001 | | | |
| 3-methyl Butanal | p < 0.001 | | | | |
| Pentanal | | p < 0.001 | | | |
| 2.3-dihydro Furan | | p < 0.001 | | | |
| Hexanal | | p < 0.001 | p < 0.001 | p < 0.001 | |
| 1,3,5-trimethyl | p < 0.001 | | | | |
| cyclohexane | | | | | |
| 2,4-dimethyl1- | | p < 0.001 | | | p < 0.001 |
| Heptene | | | | | |
| Unknown | | p < 0.001 | | | |
| 2,4-dimethyl | | | p < 0.001 | | p < 0.001 |
| Decane | | | | | |
| 4,7-dimethyl | | | p < 0.001 | | p < 0.001 |
| Undecane | | | | | |
| Unknown | | | p < 0.001 | | p < 0.001 |
| Unknown | | p < 0.001 | | | |

Urine samples

| | | | | | | |
|---|---|---|---|---|---|---|
| Hexanal | | p < 0.001 | | | | |
| 2,4-dimethyl | | | | | p < 0.001 | |
| Heptane | | | | | | |
| 4-methyl Octan | p < 0.001 | | p < 0.001 | | p < 0.001 | p < 0.001 |
| Unknown | | | p < 0.001 | | | |
| 2-ethyl 1-Hexanol | | | p < 0.001 | | | |
| Dodecane | p < 0.001 | | | | | |
| 4,7-dimethyl | p < 0.001 | | | | | |
| Undecane | | | | | | |
| 5-ethyl, 2-methyl | | | p < 0.001 | | | |
| Octane | | | | | | |
| Unknown | p < 0.001 | | | | p < 0.001 | p < 0.001 |
| Unknown | | | p < 0.001 | p < 0.001 | | |

Additional study was performed, similar to the one described in the above examples, with a different number of samples. Blood samples were obtained from 130 healthy volunteers (control, C); 32 kidney cancer (KC) patients; 25 gastric cancer (GC) patients; 22 dyspeptic patients who underwent fibrogastroscopy (FG); and 11 lung cancer (LC) patients. Urine samples were obtained from 97 healthy volunteers, 9 kidney cancer patients; 12 gastric cancer patients; 11 dyspeptic patients who underwent fibrogastroscopy; and 9 lung cancer patients.

Table 16 shows VOCs which provided significant discrimination between healthy and cancerous patients.

Table 17 summarizes different combinations of sensors, which provided efficient discrimination between healthy and cancerous patients and between different types of cancer.

TABLE 17
Chemically sensitive sensors used for discrimination between samples indicated
in the left column of the table.
Array of chemically sensitive sensors, Blood

| | | | |
|---|---|---|---|
| C vs. | 2-Naphthalenethiol | dodecanethiol GNPs | decanethiol |
| GC | GNPs | | GNPs |
| C vs | Decanethiol GNPs | 2-Naphthalenethiol | |
| KC | | GNPs | |
| C vs | Tert-dodecanethiol | | |
| LC | GNPs | | |
| FG | Tert-dodecanethiol | Diketopyrrolopyrrole- | 2- |
| vs | GNPs | anthracene (FAF) | Naphthalenethiol |
| GC | | | GNPs |
| GC | Hexyldecyl- | CB/(PUC-2S/PPUU- | |
| vs | substituted | 2S) composite | |
| KC | poly(diketopyrrolopyrrole) | | |
| GC | Decanethiol GNPs | CB/(PPMA/PEI) | 3- |
| vs | | Composite | Ethoxythiophenol |
| LC | | | GNPs |
| KC | Diketopyrrolopyrrole- | Decanethiol GNPs | Diketopyrrolopyrrole- |
| vs | naphthalene (TNT) | | benzothiadiazole |
| LC | | | (TBT) |

Array of chemically sensitive sensors, Urine

| | | |
|---|---|---|
| C vs. | Diketopyrrolopyrrole- | 4-tert- |
| GC | anthracene (FAF) | Butylbenzenethiol |
| | | GNPs |
| C vs | Tert-dodecanethiol | Diketopyrrolopyrrole- |
| KC | GNPs | anthracene (FAF) |
| C vs | Octadecanethiol GNPs | Decanethiol GNPs |
| LC | | |
| FG | Tert-dodecanethiol | 1,6-Hexanedithiol- |
| vs | GNPs | coated GNPs |
| GC | | |
| GC | Diketopyrrolopyrrole- | |
| vs | naphthalene (TNT) | |
| KC | | |
| GC | Tert-dodecanethiol | dodecanethiol GNPs |
| vs | GNPs | |
| LC | | |
| KC | Decanethiol GNPs | CB/(PPMA/PEI) |
| VS | | Composite |
| LC | | |

Portable device, Blood

| | | | | |
|---|---|---|---|---|
| C vs. | Dodecanethiol GNPs | 2-Ethylhexanethiol | | |
| GC | | GNPs | | |
| C vs | Dodecanethiol GNPs | 3-ethoxytiophenol | | |
| KC | | GNPs | | |
| C vs | Dodecanethiol GNPs | 2-Ethylhexanethiol | 3- | |
| LC | | GNPs | ethoxytiophenol | |
| | | | GNPs | |
| FG | Dodecanethiol GNPs | 2-Ethylhexanethiol | | |
| vs | | GNPs | | |
| GC | | | | |
| GC | 3-ethoxytiophenol | | | |
| vs | GNPs | | | |
| KC | | | | |
| GC | 4-tert methyl- | 3-ethoxytiophenol | | |
| vs | benzenethiol GNPs | GNPs | | |
| LC | | | | |
| KC | 2-Ethylhexanethiol | Decanethiol GNPs | 3- | 4-chlorobenzene- |
| vs | GNPs | | ethoxytiophenol | methanethiol |
| LC | | | GNPs | GNPs |

Portable device, Urine

| | | | | |
|---|---|---|---|---|
| C vs. | Dodecanethiol GNPs | 2-Ethylhexanethiol | 4-tert methyl- | |
| GC | | GNPs | benzenethiol | |
| | | | GNPs | |
| C vs | 4-tert methyl- | Decanethiol GNPs | 3- | 4-chlorobenzene- |
| KC | benzenethiol GNPs | | ethoxytiophenol | methanethiol |
| | | | GNPs | GNPs |
| C vs | Dodecanethiol GNPs | 3-ethoxytiophenol | | |
| LC | | GNPs | | |
| FG | Hexanethiol GNPs | | | |
| vs | | | | |
| GC | | | | |
| GC | Dodecanethiol GNPs | 3-ethoxytiophenol | | |
| vs | | GNPs | | |
| KC | | | | |
| GC | 2-Ethylhexanethiol | Decanethiol GNPs | 3- | |
| vs | GNPs | | ethoxytiophenol | |
| LC | | | GNPs | |
| KC | 3-ethoxytiophenol | 4-chloroben- | | |
| vs | GNPs | zenemethanethiol | | |
| LC | | GNPs | | |

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-43. (canceled)

44. A method of diagnosing cancer in a test subject, comprising contacting a portable device with a blood sample and/or a urine sample obtained from the test subject, wherein the portable device comprises an array of chemically sensitive sensors comprising a material selected from the group consisting of conductive nanostructures coated with an organic coating, a conducting polymer and a conductive polymer composite, and analyzing output signals of the chemically sensitive sensors upon exposure of the array to the blood sample and/or the urine sample;
wherein contacting comprises drawing an aliquot of a headspace of the blood sample and/or an aliquot of a headspace of the urine sample into the device and exposing the array to each aliquot individually;
wherein analyzing comprises using a model based on a database of response patterns of the array of the chemically sensitive sensors to control samples comprising blood samples and/or urine samples obtained from patients having the cancer and healthy subjects; and
wherein the cancer is selected from the group consisting of kidney cancer, gastric cancer, lung cancer, breast cancer, pancreatic cancer, ovarian cancer, colon cancer, head and neck cancer, prostate cancer, and combinations thereof.

45. The method of claim 44 comprising contacting the portable device with a blood sample and a urine sample and analyzing output signals of the chemically sensitive sensors upon exposure of the array to the blood sample and the urine sample.

46. The method of claim 44, wherein the conductive nanostructures coated with an organic coating are selected from gold nanoparticles (GNPs) coated with a thiol or a disulfide and single walled carbon nanotubes (SWCNTs) coated with polycyclic aromatic hydrocarbon (PAH).

47. The method of claim 46, wherein the thiol is selected from the group consisting of dodecanethiol, 4-tert methyl-benzenethiol, 2-ethylhexanethiol, decanethiol, 4-chlorobenzenemethanethiol, 3-ethoxytiophenol, hexanethiol, octadecanethiol, and combinations thereof; or wherein the polycyclic aromatic hydrocarbon comprises hexa-perihexabenzocoronene or a derivative thereof.

48. The method of claim 44, wherein the conducting polymer is selected from the group consisting of diketopyrrolopyrrole-naphthalene copolymer (PDPP-TNT), polydiketopyrrolopyrrole, polyaniline (PANT), polythiophene, poly(3,4-ethylenedioxythiophene)-poly(styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof.

49. The method of claim 44, wherein the conductive polymer composite comprises a polymer selected from the group consisting of a disulfide polymer, a methacrylate polymer, a polyethyleneimine polymer, combinations and derivatives thereof, wherein said polymer is mixed with a carbon powder; or wherein the conductive polymer composite is selected from the group consisting of carbon black/poly(propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

50. The method of claim 44, wherein the array comprises eight chemically sensitive nanosensors comprising gold nanoparticles coated with dodecanethiol, 4-tert methyl-benzenethiol, 2-ethylhexanethiol, decanethiol, 4-chlorobenzenemethanethiol, 3-ethoxytiophenol, hexanethiol, and octadecanethiol.

51. The method of claim 44, wherein the array of chemically sensitive sensors is sealed within the portable device from the external atmosphere; or wherein the portable device further comprises a sample inlet, a cannula, and a pipe, wherein the pipe is connected to the cannula at one end and to the sample inlet at another end.

52. The method of claim 51, wherein drawing an aliquot of a headspace of the blood sample and an aliquot of a headspace of the urine sample comprises inserting the cannula into a vial comprising the respective sample and pumping the headspace into the portable device.

53. The method of claim 52, wherein pumping rate ranges from about 30 μl/s to about 3300 μl/s and/or wherein pumping is performed for a period ranging from about 0.5 s to about 5 s; or wherein the array is exposed to the aliquot of the headspace for a period ranging from about 5 s to about 120 s.

54. The method of claim 44, wherein the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal.

55. The method of claim 44, wherein the cancer is selected from the group consisting of kidney cancer, gastric cancer, and lung cancer.

56. The method of claim 44, wherein the model is developed by using at least one algorithm selected from the group consisting of random forest (RF) clustering, Ward's minimum variance method, discriminant function analysis (DFA), artifical neural network (ANN) algorithm, support vector machine (SVM), pricipal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA).

57. A method of diagnosing cancer in a test subject, comprising exposing an array of chemically sensitive sensors comprising a material selected from the group consisting of conductive nanostructures coated with an organic coating, a conducting polymer and a conductive polymer composite, to a blood sample and a urine sample obtained from the test subject and analyzing output signals of the chemically sensitive sensors upon exposure of the array to the blood sample and urine sample;
wherein the array comprises gold nanoparticles coated with octadecanethiol;
wherein analyzing comprises using a model based on a database of response patterns of the array of the chemically sensitive sensors to control samples comprising blood and urine samples obtained from patients having the cancer and healthy subjects; and
wherein the cancer is selected from the group consisting of kidney cancer, gastric cancer, lung cancer, breast cancer, pancreatic cancer, ovarian cancer, colon cancer, head and neck cancer, prostate cancer, and combinations thereof.

58. The method of claim 57, wherein the array further comprises gold nanoparticles coated with an organic coating selected from the group consisting of tert-dodecanethiol, butanethiol, 4-cholorobenzenemethanthiol, 4-tert butylbenzenethiol, 2-naphthalenethiol, 2-nitro-4-(trifluoromethyl)benzenethiol, dodecanethiol, decanethiol, 3-ethoxythiophenol, benzylmercaptan, hexanethiol, 2-ethylhexanethiol, 1,6-hexanedithiol, butanethiol, dibutyl disulfide, and combinations thereof; or wherein the array further comprises single walled carbon nanotubes (SWCNTs) coated with a polycyclic aromatic hydrocarbon (PAH) or a derivative thereof selected from the group consisting of hexa-peri-hexabenzocoronene (HBC) molecules that can be unsubstituted or substituted by any one of methyl ether, 2-ethyl-hexyl (HBC-C6,2), 2-hexyldecyl (HBC-C10,6), 2-decyltetradecyl (HBC-C14,10), and dodecyl (HBC-C12); or wherein the array further comprises a conducting polymer selected from the group consisting of diketopyrrolopyrrole-naphthalene copolymer (PDPP-TNT), polydiketopyrrolopyrrole, polyaniline (PANT), polythiophene, poly(3,4-ethylenedioxythiophene)-poly(styrene-sulfonate) (PEDOT:PSS), polypyrrole, diketopyrrolopyrrole-anthracene copolymer (PDPP-FAF), diketopyrrolopyrrole-benzothiadiazole (TBT), and derivatives and combinations thereof; or wherein the array further comprises a conductive polymer composite selected from the group consisting of a disulfide polymer, a methacrylate polymer, a polyethyleneimine polymer, combinations and derivatives thereof, wherein said polymer is mixed with a carbon powder; or wherein the array further comprises carbon black/poly(propylene-urethaneureaphenyl-disulfide) composite, carbon black/poly(propylene-urethaneureaphenyl-disulfide)/poly(urethane-carboxyphenyl-disulfide) composite, and carbon black/poly(2-hydroxypropyl methacrylate)/polyethyleneimine composite.

59. The method of claim 57, wherein the cancer is selected from the group consisting of kidney cancer, gastric cancer, and lung cancer; or wherein the model is developed by using at least one algorithm selected from the group consisting of random forest (RF) clustering, Ward's minimum variance method, discriminant function analysis (DFA), artificial neural network (ANN) algorithm, support vector machine (SVM), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA); or wherein the array of the chemically sensitive sensors is configured to detect at least five volatile organic compounds (VOCs) present in the headspace of the blood sample and/or the headspace of the urine sample, wherein the VOCs are selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, and 3-methyl butanal; or wherein exposing the array to each one of the blood sample and the urine sample provides enhanced accuracy, sensitivity and/or specificity of the diagnosing as compared to exposing to only one of said blood sample and urine sample.

60. A method of diagnosing cancer in a test subject, comprising measuring and analyzing levels of a set of volatile organic compounds (VOCs) in a blood sample and a urine sample obtained from the test subject,
wherein the set of VOCs comprises at least five VOCs selected from the group consisting of 2-methyl 2-propanol, butanal, 2,4,4-trimethyl 1-pentene, butyl alcohol, 2,3,5-trimethyl hexane, 4-heptanone, 2-heptanone, heptanal, 1-octene-3-ol, 2-pentyl furan, 3-ethyl-3-methylheptane, 2-methyl-3-oxo-3-(2-pyridinyl)propanoic acid ethyl ester, 2,7,10-trimethyl-dodecane, tetradecane, 2-pentanone, dimethyl disulfide, 3-hexanone, 3-heptanone, 5-methyl 3-hexanone, allyl isothiocyanate, dimethyl trisulfide, 2,3-octanedione, 2,6-dimethyl nonane, 1-(3-methylphenyl) ethenone, p-cresol, 2-nonanone, 4,5-dimethyl nonane, 3-phenyl-2-pentene, pentyl benzene, menthol, 1-methyl-1-butenyl benzene, carvone, pentadecane, 2,3,5,8-tetramethyl-decane, and 2,4-bis(1,1-dimethylethyl) phenol, 4,5 dimethyl nonane, hexane, dodecane, 5-ethyl 2-methyl octane, 2,4 dimethyl decane, hexanal, pentanal, 2,3 dihydrofuran, dodecane, 3-methyl butanal;
wherein analyzing comprises using a model based on a database of levels of the set of VOCs in control samples comprising blood and urine samples obtained from patients having the cancer and healthy subjects; and
wherein the cancer is selected from the group consisting of kidney cancer, gastric cancer, lung cancer, breast cancer, pancreatic cancer, ovarian cancer, colon cancer, head and neck cancer, prostate cancer, and combinations thereof.

61. The method of claim 60, wherein the set of VOCs comprises at least five VOCs selected from the group consisting of hexane, 3-methyl butanal, pentanal, 2.3-dihydrofuran, hexanal, 1,3,5-trimethyl cyclohexane, 2,4-dimethyl1-heptene, 2,4-dimethyl decane, 4,7-dimethyl undecane, 2,4-dimethyl heptane, 4-methyl octane, 2-ethyl 1-hexanol, dodecane, 5-ethyl,2-methyl octane, and combinations thereof; or wherein the set of VOCs comprises at least five VOCs selected from the group consisting of 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydrofuran, 2,3,5 trimethyl hexane, 2-pentyl furan, and 3-methyl butanal; or wherein the set of VOCs comprises 2,3,5,8-tetramethyl decane, 3-hexanone, p-cresol, pentadecane, 4,5 dimethyl nonane, hexane, 2,6 dimethyl nonane, 2-nonanone, 1-(3-methyl phenyl) ethenone, 3-phenyl 2-pentene, 4-heptanone, 2-heptanone, dodecane, 2-heptanone, 2-methyl 2-propanol, 5-ethyl 2-methyl octane, heptanal, 1-octene-3-ol, 3-ethyl 3-methylheptane, tetradecane, 2,4 dimethyl decane, hexanal, pentanal, 2,3 dihydrofuran, 2,3,5 trimethyl hexane, 2-pentyl furan, and 3-methyl butanal; or wherein the set of VOCs comprises at least five VOCs selected from the group consisting of hexane, 3-methyl butanal, pentanal, 2.3-dihydrofuran, hexanal, 1,3,5-trimethyl cyclohexane, 2,4-dimethyl1-heptene, 2,4-dimethyl decane, 4-methyl octane, and 5-ethyl,2-methyl octane; or wherein the set of VOCs comprises at least five VOCs selected from the group consisting of 3-methyl butanal, pentanal, hexanal, 2,3-dihydrofuran, 2,4-dimethyl decane, dodecane, 2-ethyl hexanol, 5-ethyl-2-methyl octane.

62. The method of claim 60, wherein the model is developed by using at least one algorithm selected from the group consisting of random forest (RF) clustering, Ward's minimum variance method, discriminant function analysis (DFA), artifical neural network (ANN) algorithm, support vector machine (SVM), pricipal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA); or wherein measuring the levels of a set of VOCs comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), and Proton Transfer Reaction Mass-Spectrometry (PTR-MS).

63. The method of claim 60, wherein the cancer is selected from the group consisting of kidney cancer, gastric cancer, and lung cancer.

64. A portable device configured to come into contact with a blood sample and/or a urine sample obtained from a test subject, comprising an array of eight chemically sensitive nanosensors comprising gold nanoparticles coated with dodecanethiol, 4-tert methyl-benzenethiol, 2-ethylhexanethiol, decanethiol, 4-chlorobenzenemethanethiol, 3-ethoxytiophenol, hexanethiol, and octadecanethiol.

65. The portable device of claim 64, wherein the array of chemically sensitive sensors is sealed within the portable device from the external atmosphere; or wherein the portable device further comprises a sample inlet, a cannula, and a pipe, wherein the pipe is connected to the cannula at one end and to the sample inlet at another end.
